(19) United States
(12) Patent Application Publication
(10) Pub. No.: US 2013/0123619 A1
(43) Pub. Date: May 16, 2013

(54) HEMODYNAMIC PRESSURE SENSOR TEST SYSTEM AND METHOD

(71) Applicant: Leon T. Griggs, Minnetonka, MN (US)

(72) Inventor: Leon T. Griggs, Minnetonka, MN (US)

(73) Assignee: ACIST MEDICAL SYSTEMS, INC., Eden Prairie, MN (US)

(21) Appl. No.: 13/464,715

(22) Filed: May 4, 2012

Related U.S. Application Data
(60) Provisional application No. 61/482,440, filed on May 4, 2011.

(51) Int. Cl. G01L 7/00; A61M 5/00

(52) U.S. Cl. CPC G01L 7/00; A61M 5/007
USPC 600/432; 73/700

(57) ABSTRACT

A pressure sensor suitable for use in a powered contrast injector system may be tested to help validate the operability and/or integrity of the sensor. In some examples, the pressure sensor may be tested by generating a pressure pulse in a fluid line fluidly connected to the pressure sensor so as to generate a first pressure reading. A high pressure fluid at a pressure above a maximum operating pressure of the pressure sensor may be conveyed through a valve fluidly connected to the pressure sensor. Subsequent to conveying the high pressure fluid through the valve, the pressure sensor may again be tested by generating a pressure pulse in the fluid line fluidly connected to the pressure sensor so as to generate a second pressure reading. In some examples, the first pressure reading is compared to the second pressure reading to determine whether the pressure sensor has passed or failed.

This application claims the benefit of U.S. Provisional Patent Application No. 61/482,440, filed May 4, 2011, the entire contents of which are incorporated herein by reference.

### TECHNICAL FIELD

This disclosure relates to pressure sensors and, more particularly, to pressure sensors associated with medical fluid injection systems.

### BACKGROUND

Angiography is a procedure used in the diagnosis and treatment of cardiovascular conditions that include abnormalities or restrictions in blood vessels of humans or animals. During angiography, radiographic contrast media is injected through a catheter into a vein or artery, which then passes to vascular structures in fluid communication with the vein or artery. When X-rays are passed through the region of the body into which the contrast media is injected, the X-rays are absorbed by the contrast media, providing radiographic images of the desired vascular structure(s). The images can be recorded, stored, and/or displayed on a monitor.

The radiographic images generated through angiography can be used for many purposes. One common use for radiographic imaging is to diagnose various conditions related to a patient's vasculature. Radiographic images can also be used for therapeutic procedures such as angioplasty, where a balloon is inserted into a vascular system and inflated to open a stenosis.

When used, contrast media is typically injected into a catheter by an automated injection system. While the apparatus for injecting the contrast media can vary, most systems include a syringe operatively connected with the catheter. The injector includes a syringe chamber that houses a syringe, which can typically be reused several times. The injector also includes a ram that is reciprocally moveable within the syringe chamber. The contrast media is suctioned into the syringe when the ram is moved to create a partial vacuum within the syringe. A reversal of the ram direction first forces air out of the syringe and then delivers the contrast media to the catheter at a rate and volume determined by the speed of movement of the ram.

Additionally, angiography can include the injection of fluids other than the contrast media. For example, a saline flush and/or the injection of fluid medications may be desired. Accordingly, injectors can include multiple syringe chambers for housing multiple syringes, with the injector having a ram for each syringe chamber. These additional syringe chambers and rams can function the same way as discussed above, with the only difference being that fluids other than contrast media are suctioned into and delivered out of the respective syringes. An injector with multiple syringes is described in U.S. patent application Ser. No. 12/094,009 (Publication No. 2009/0149743), titled Medical Fluid Injection System, which is assigned to the assignee of the present application and is hereby incorporated by reference in its entirety.

In some applications, a patient's hemodynamic pressure may be monitored during an injection procedure. For example, during angiography, a health care provider may record the intravascular and intra-cardiac pressures of the patient between injections of high pressure contrast media. The health care provider may look for pressure values falling within the general range of −1 psi to +6 psi (−51.7 mmHg to 310 mmHg) to confirm the hemodynamic health of the patient.

To monitor the hemodynamic pressure of a patient during an injection procedure, the patient may be connected to a pressure sensor that is in fluid communication with a catheter inserted into the patient. The catheter connected to the pressure sensor may also be used to inject fluids into the patient. For example, contrast injection media may be delivered through the catheter to the patient at a high pressure (e.g., a pressure around 1200 pounds per square inch), while saline is subsequently delivered to the patient through the same catheter at a comparatively lower pressure (e.g., less than 125 pounds per square inch). Depending on the design of the pressure sensor, the pressure in the catheter during injection of contrast media may be higher than the pressure rating of the pressure sensor. Accordingly, the pressure sensor may be shielded from high pressures developed during contrast media injection by a selective fluid delivery valve. The selective fluid delivery valve may close fluid communication between the pressure sensor and the catheter during comparatively high pressure contrast media injection and open fluid communication between the pressure sensor and the catheter when high pressure contrast media is not being injected through the catheter. If the selective fluid delivery valve does not adequately seal fluid communication between the pressure sensor and the catheter during a high pressure contrast media injection, the pressure sensor may be damaged, rendering subsequent pressure readings from the pressure sensor unreliable.

### SUMMARY

In general, this disclosure is directed to systems and techniques for testing a medical pressure sensor to help ensure the consistency of pressure readings generated by the pressure sensor before and after high pressure fluid injections. In some examples as described herein, pressure pulses are generated in a fluid line fluidly connected to the pressure sensor before a high pressure fluid injection to generate a first set of pressure sensor measurements. High pressure fluid such as high pressure contrast injection media is subsequently passed through a selective fluid delivery valve connected to the pressure sensor. If the selective fluid delivery valve is operating as intended, the valve may shield the pressure sensor from the high pressure fluid by closing fluid communication between the high pressure fluid and the pressure sensor. However, if the integrity of the selective fluid delivery valve is compromised, the pressure sensor may be exposed to the high pressure fluid. Depending on the design of the specific pressure sensor, exposure to high pressure fluid may damage the pressure sensor. Accordingly, after passing the high pressure fluid through the selective fluid delivery valve, pressure pulses may again be generated in the fluid line fluidly connected to the pressure sensor to generate a second set of pressure sensor measurements. The first set of pressure measurements may be compared to the second set of pressure measurement to determine if there has been any change in the performance (e.g., sensitivity, pressure readings) of the pressure sensor before and after the introduction of the high pressure fluid. A change in pressure readings before and after the introduction of the high pressure fluid may indicate that there is a problem with the pressure sensor, the selective fluid delivery valve, and/or an assembly of the pressure sensor fluidly connected to the selective fluid delivery valve.

In one example, a powered injection system configured to deliver contrast injection media to a patient is described. The powered injection system includes a control panel to receive input from a user, an injector control system having a pressure sensor testing control system in electrical communication with the control panel, a syringe driven by a power source in electrical communication with the control panel, a first fluid reservoir in fluid communication with the syringe, a syringe outlet tube in fluid communication with the syringe, a second fluid reservoir, and a tubing system to deliver a first fluid from the first fluid reservoir to a patient line and to deliver a second fluid from the second fluid reservoir to the patient line. The example states that the power injection system also includes a selective fluid delivery valve in fluid communication with the syringe outlet tube and the second fluid reservoir and having a valve outlet port in fluid flow communication with the patient line, the selective fluid delivery valve being selectively positionable to provide a fluid flow path between the syringe outlet tube and the valve outlet port or between the second fluid reservoir and the valve outlet port. In addition, the example specifies that the power injection system includes a pressure sensor configured to monitor a homodynamic pressure of a patient, the pressure sensor being in fluid flow communication with the patient when the selective fluid delivery valve is positioned to provide fluid flow communication between the second fluid reservoir and the patient line and not being in fluid flow communication with the patient when the selective fluid delivery valve is positioned to provide fluid flow communication between the first fluid reservoir and the patent line, the pressure sensor being in electrical communication with the injector control system. According to the example, the power injection system also includes a pressure inducer in electrical communication with the injector control system, the pressure inducer being positioned to induce a pressure on the tubing system when the pressure sensor is in fluid flow communication with the patient line to generate a test pressure signal to test an operability of the pressure sensor.

In another example, a powered injection system is described that includes an injector that includes a first syringe chamber in fluid communication with a tubing system and an injector control system that includes a processor, the injector control system including a testing control system. According to the example, the testing control system includes a pressure inducer control module, a pressure sensor data receiving module, and a testing protocol module, each of which may be stored on a computer readable medium. The pressure inducer control module may, when executed by the processor, cause the processor to generate a signal to activate the pressure inducer control module to induce a pressure on a hemodynamic pressure sensor in fluid communication with the tubing system. The pressure sensor data receiving module may, when executed by the processor, cause the processor to collect an induced pressure signal from the hemodynamic pressure sensor. The testing protocol module may, when executed by the processor, cause the processor to determine whether the hemodynamic pressure sensor is operable or inoperable based on an evaluation of the induced pressure signal received by the pressure sensor data receiving module.

In another example, a test system for testing a hemodynamic pressure sensor is described. The test system includes a pressure inducer, a pressure generator, and a testing control system. According to the example, the pressure inducer is configured to induce pressure changes to a tubing system in fluid communication with the hemodynamic pressure sensor. The pressure generator is configured to power the pressure inducer. In addition, the testing control system is in electrical communication with the hemodynamic pressure sensor, the pressure inducer, and the pressure generator.

In another example, a method of testing a hemodynamic pressure sensor is described. The method includes initiating a testing control system, initiating a pressure generator, and initiating a pressure inducer to induce pressure to a tubing system associated with a hemodynamic pressure sensor. The example method also includes receiving a signal from the hemodynamic pressure sensor, testing the signal, and determining if the hemodynamic pressure sensor passes or fails.

In another example, a method is described that includes generating at least one pressure pulse in a fluid line fluidly connected to a medical pressure sensor so as to generate a first pressure reading, the medical pressure sensor being fluidly connected to a valve that is configured to shield the medical pressure sensor from a high pressure fluid injection. The method also includes conveying high pressure fluid through the valve fluidly connected to the medical pressure sensor, the high pressure fluid being at a pressure above a maximum operating pressure of the medical pressure sensor. In addition, the method includes, subsequent to conveying the high pressure fluid, generating at least one pressure pulse in the fluid line fluidly connected to the medical pressure sensor so as to generate a second pressure reading. The method further includes comparing the first pressure reading to the second pressure reading and determining based on the comparison whether the medical pressure sensor has passed or failed.

The details of one or more examples are set forth in the accompanying drawings and the description below. Other features, objects, and advantages will be apparent from the description and drawings, and from the claims.

### BRIEF DESCRIPTION OF DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIGS. 1A and 1B are perspective views of an example injector system in accordance with embodiments of the invention.

FIG. 1C is a schematic diagram of an injector system and tubing system in accordance with an embodiment of the invention.

FIGS. 2A and 2B are perspective views of example disposable fluid connections that may be used in the example injection system of FIGS. 1A and 1B.

FIG. 3 is a cross-sectional view of an example fluid delivery valve that may be used with the example injection system of FIGS. 1A and 1B.

FIG. 4 is a cross-sectional view of the example fluid delivery valve of FIG. 3 during a contrast injection operation.

FIG. 5 is a cross-sectional view of another example fluid delivery valve that may be used with the example injection system of FIGS. 1A and 1B.

FIG. 6 is a cross-sectional view of the example fluid delivery valve of FIG. 5 during a contrast injection operation.

FIG. 7 is a schematic diagram of a test system in accordance with an embodiment of the invention.

FIG. 8 is a conceptual block diagram of an injector control system with a testing control system in accordance with embodiments of the invention.

FIG. 9 is a flow chart of an illustrative method in accordance with embodiments of the invention.

FIG. 10 is conceptual block diagram of an example pressure inducer in accordance with an embodiment of the invention.

FIG. 11 is a conceptual block diagram of another example pressure inducer in accordance with an embodiment of the invention.

FIG. 12 is a flow chart of another illustrative method in accordance with embodiments of the invention.

FIGS. 13-15 are plots illustrating example pressures that may be measured by a pressure according to the example method of FIG. 12.

FIG. 16 is a schematic diagram of a test system in accordance with an embodiment of the invention.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention. Examples of constructions, materials, dimensions, and manufacturing processes may be provided for selected elements, and all other elements employ that which is known to those of skill in the field of the invention. Those skilled in the art will recognize that many of the examples provided have suitable alternatives that can be utilized.

In general, this disclosure is directed to systems and methods for testing a hemodynamic pressure sensor suitable for use in a powered contrast injector system. In some examples, the hemodynamic pressure sensor is tested while associated with or incorporated in the powered contrast injector system. In other examples, the hemodynamic pressure sensor is tested prior to being associated with or incorporated in the powered contrast injector system, e.g., after manufacture or receipt from a manufacturer but prior to use with the power contrast injector system. Regardless, testing the hemodynamic pressure sensor may help validate the operability and/or integrity of the hemodynamic pressure sensor (and/or other hardware associated with the hemodynamic pressure sensor such as a selective fluid delivery valve), helping to ensure that the hemodynamic pressure sensor provides reliable data during subsequent use.

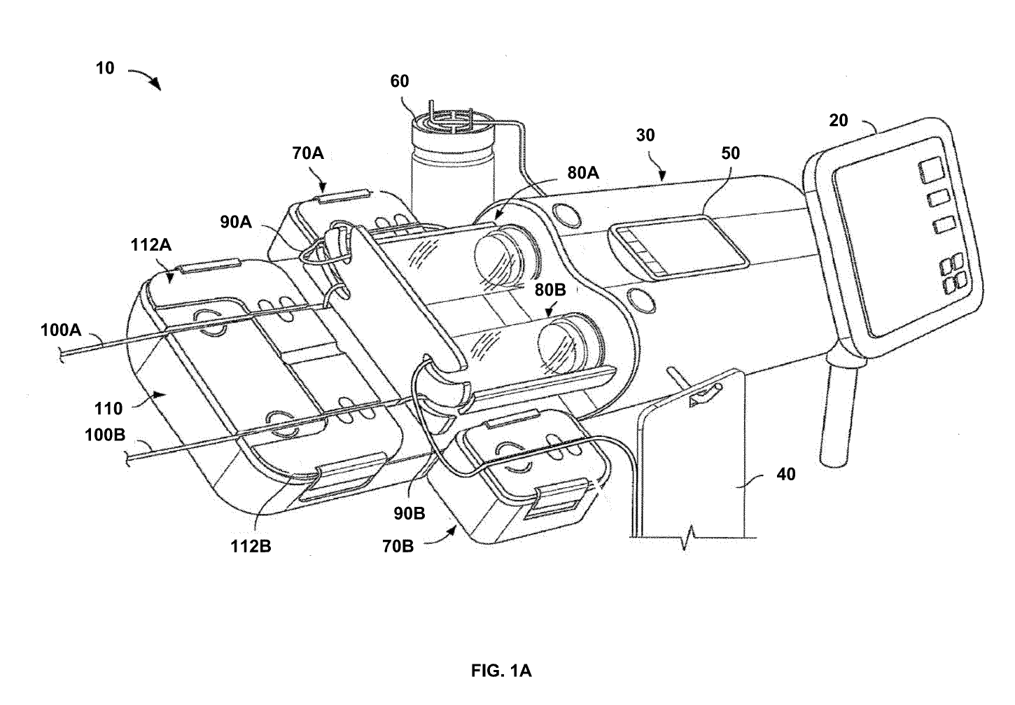

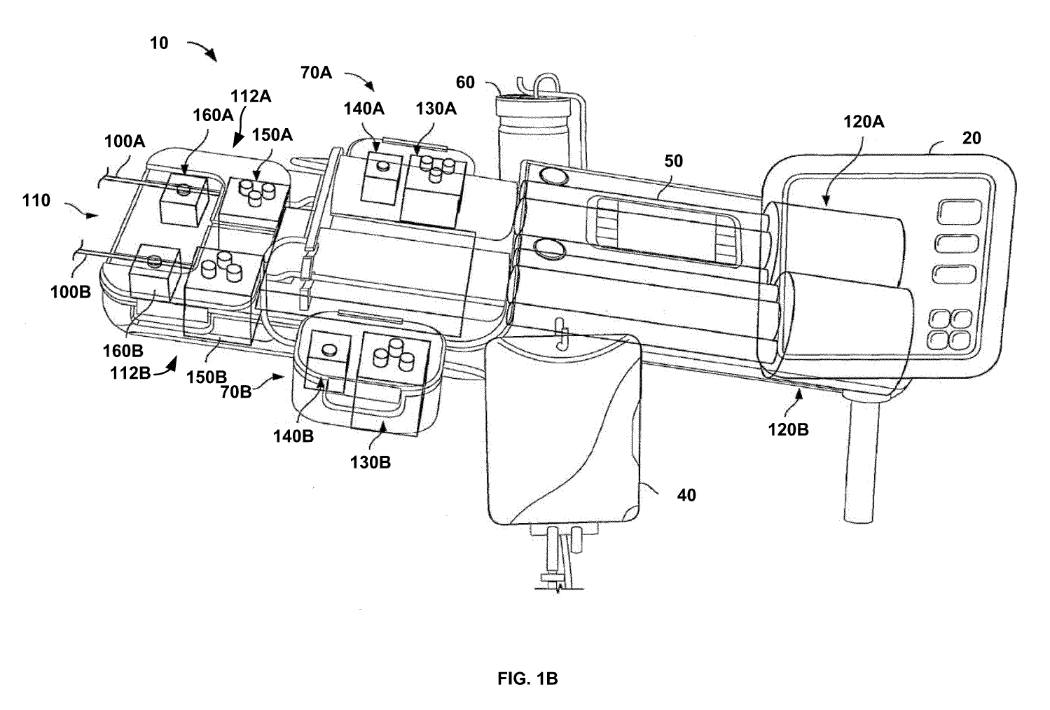

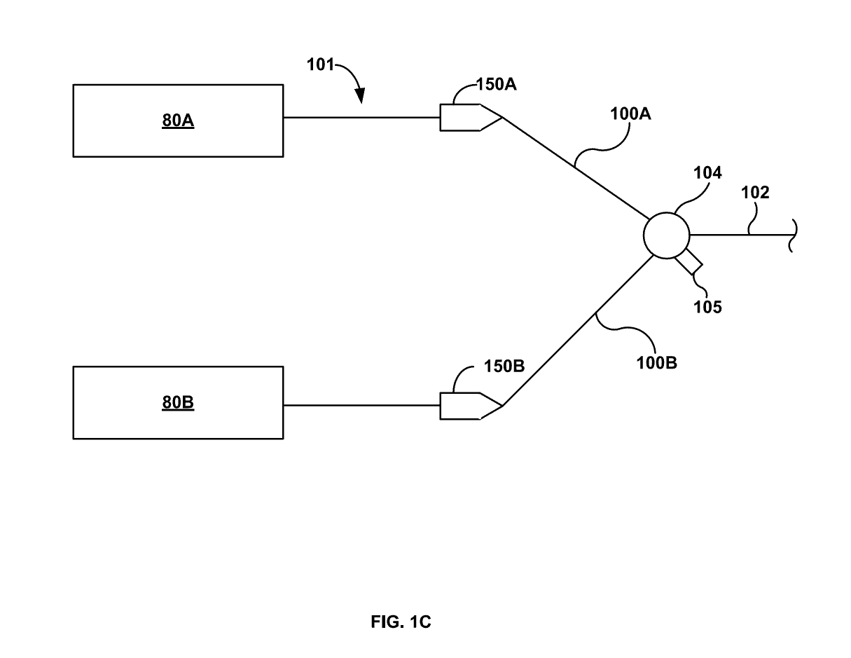

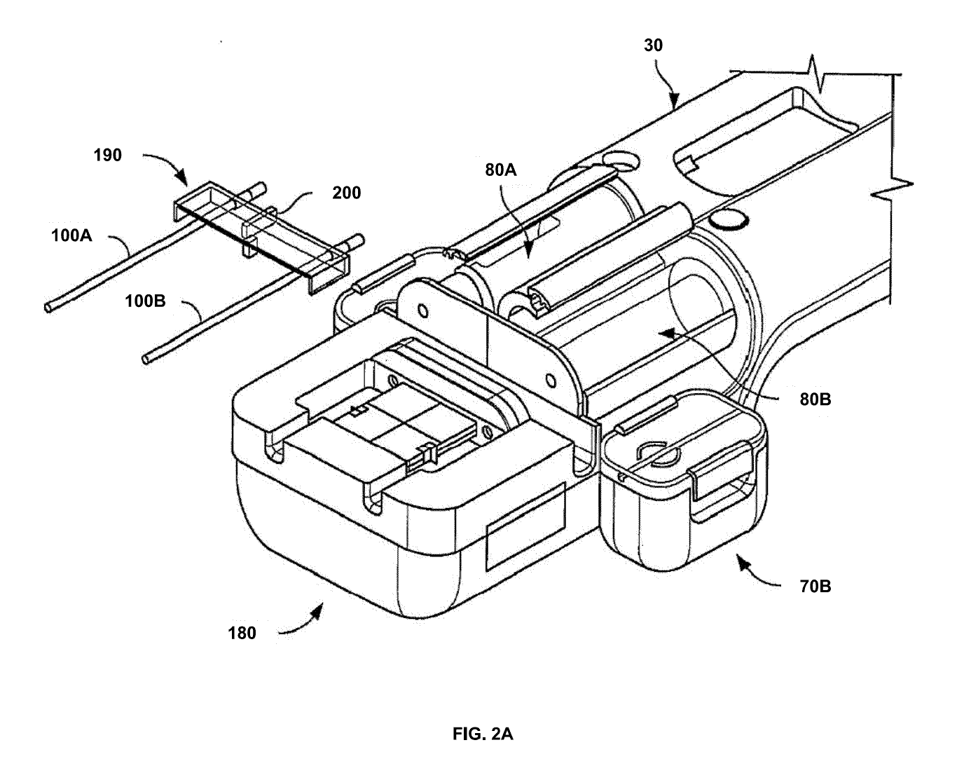

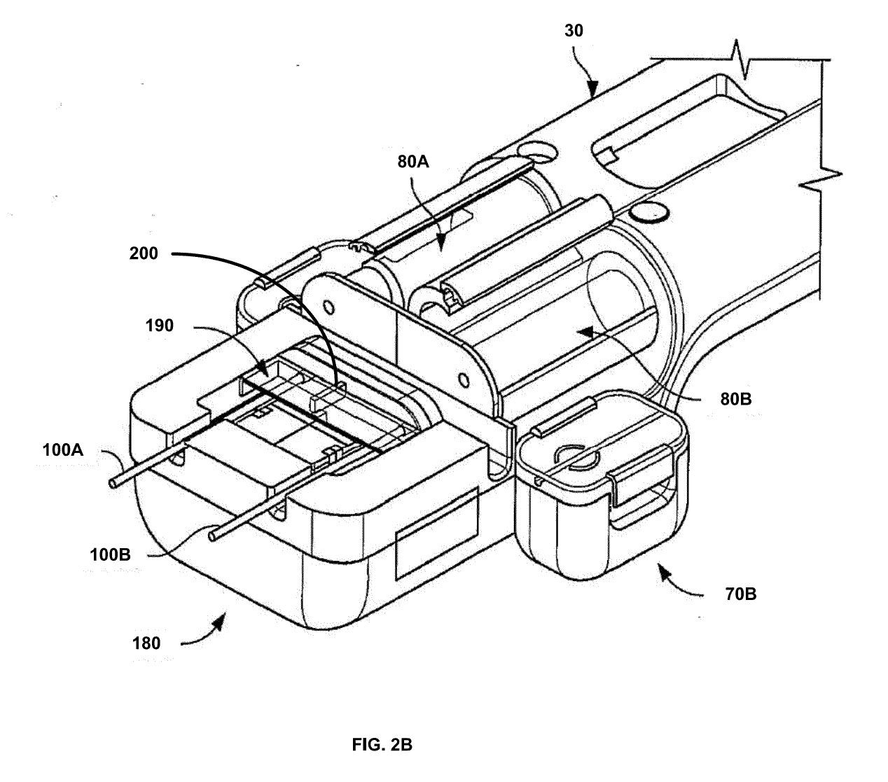

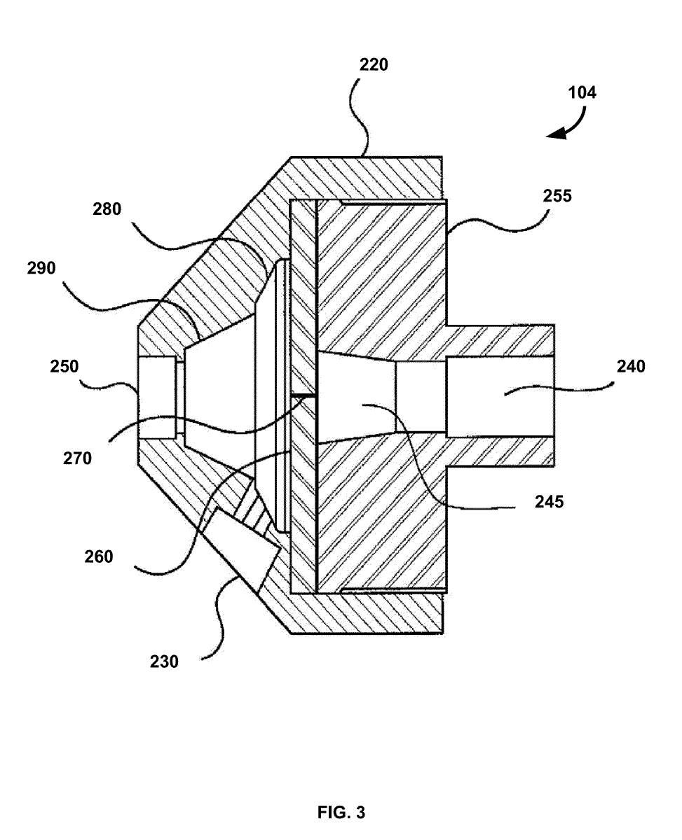

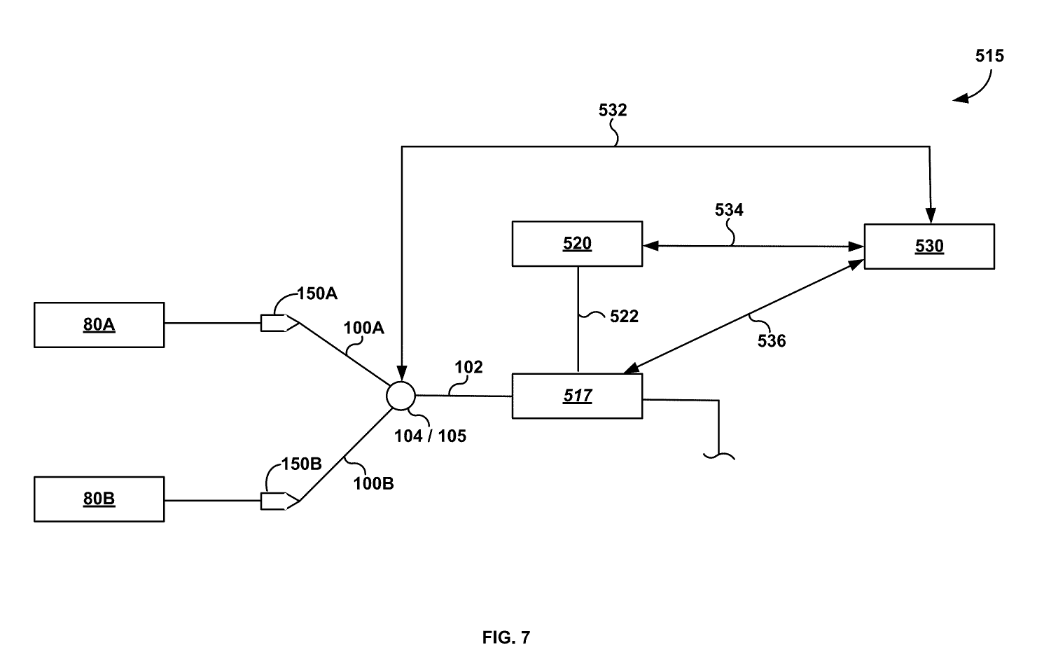

Example systems and methods for testing a hemodynamic pressure sensor are described in greater detail with respect to FIGS. 7-16. Further, example selective fluid delivery valves that may be fluidly connected to a hemodynamic pressure sensor are described in greater detail with respect to FIGS. 3-6. However, an example system that includes a powered contrast injector system and a pressure sensor will first be described with reference to FIGS. 1A-1C and 2A-2B.

An embodiment of a contrast injection system in accordance with some examples of the disclosure is shown in FIGS. 1A and 1B. FIGS. 1A and 1B are views of an exemplary dual-syringe contrast injection system. FIG. 1A is a perspective view of the injection system 10. FIG. 1B is a view of the injection system 10 that shows various internal components. The injection system 10 shown in these figures is a dual-syringe system that includes a main control panel 20, an injection head 30, and an power supply (not shown). The power supply may be an electrical power supply that drives injection head 30. A first reservoir of medical fluid 60 is attached to the injection head 30, and a second reservoir of medical fluid 40 is also attached to the injection head 30. In one embodiment, the first reservoir 60 comprises a bottle of contrast media, and the second reservoir 40 comprises a bag of sterile diluent, such as saline.

In the example of injection system 10, the injection head 30 includes various sub-components. For example, the injection head 30 includes a small control panel 50, first and second syringe/plunger assemblies 80A and 80B, first and second valve/air detection assemblies 70A and 70B, and assembly 110. The first and second syringe/plunger assemblies 80A and 80B each include a syringe chamber that houses a syringe and a plunger that is axially moveable through the syringe. Assembly 110 is positioned at a discharge end of first and second syringe/plunger assemblies 80A and 80B, and, as described in greater detail below with respect to FIG. 1B, houses various components of the injection system 10.

During operation, the injection system 10 can draw fluid from the first reservoir 60 into the first syringe/plunger assembly 80A via tubing 90A by actuating a plunger to create a partial vacuum within the first syringe (i.e., by drawing the plunger back into injection head 30). The injection system 10 can also draw fluid from the second reservoir 40 into the second/plunger assembly 80B via tubing 90B by actuating a second plunger to create a partial vacuum within the second syringe. By reversing the direction of plunger travel, injection system 10 can subsequently eject fluid out of the first syringe and/or second syringe of injection system 10 and through tube 100A and 100B, respectively, into a patient's body. Injection system 10 can control the rate, volume, and source of fluid delivered to the patient, e.g., by controlling which plunger is actuated and the speed at which the plunger moves through a syringe.

As briefly noted above, injection system 10 includes a first valve/air detection assembly 70A and a second valve/air detection assembly 70B. In the embodiment shown, the assembly 110 of injection system 10 includes third and fourth valve/air detection assemblies 112A and 112B. In the example of FIGS. 1A and 1B, first valve/air detection assembly 70A is configured to control fluid flow through tubing 90A, and second valve/air detection assembly 70B is configured to control fluid flow through tubing 90B. Further, third valve/air detection assembly 112A is configured to control fluid flow through tubing 100A, while fourth valve/air detection assembly 112B is configured to control fluid flow through tubing 100B.

In some embodiments, the assembly 80A is capable of expelling contrast media into contrast media output tubing 100A, and assembly 80B is capable of expelling diluent into diluent output tubing 100B. In such embodiments, the contrast media output tubing 100A runs through a third valve/air detection assembly 112A, and the diluent output tubing 100B runs through a fourth valve/air detection assembly 112B.

FIG. 1B shows an internal view of certain components of injection system 10. Specifically, FIG. 1B shows two motor/actuator assemblies 120A and 120B. Each motor in an assembly drives a linear actuator. Each linear actuator in turn drives a plunger of one of the syringes. For example, the linear actuator in the motor/actuator assembly 120A can drive a plunger in the syringe assembly 80A, and the linear actuator in the motor/actuator assembly 120B can drive a plunger in the syringe assembly 80B. An individual plunger is capable of moving within a syringe barrel in either a forward or rearward direction. When moving in a forward direction, the plunger injects liquid into the patient line (e.g., patient line 102 in FIG. 1C). For example, when a plunger within the syringe assembly 80A moves in a forward direction (to the left in FIG. 1B), the plunger is capable of injecting liquid into the output tubing 100A. When moving in a rearward direction (to the right in FIG. 1B), the plunger is capable of filling liquid into the syringe assembly 80A from the first reservoir 60.

As shown in greater detail in FIG. 1B, the assembly 70A comprises a valve 130A and an air detector 140A. Likewise, the assembly 70B comprises a valve 130B and an air detector 140B. The air detectors 140A and 140B function to detect any air in the tubes of the system. In one embodiment, the valves 130A and 130B comprise pinch valves. Each valve 130A and 130B may be opened or closed by the injection system 10 to control fluid communication leading to syringe assemblies 80A and 80B via tubing 90A and 90B (FIG. 1A), respectively. For example, the injection system 10 may open the valve 130A to fill fluid from the first reservoir 60 into the syringe assembly 80A. The injection system 10 may also open the valve 130B to fill fluid from the second reservoir 40 into the syringe assembly 80B. The injection system 10 may close the valves 130A and/or 130B during injection of fluid. For example, the injection system 10 may close the valve 130A when the syringe assembly 80A is being used to inject fluid from the assembly 80A into the output tubing 100A.

Also as shown in the embodiment of FIG. 1B, the assemblies 112A and 112B include output valves 150A and 150B, as well as output air detectors 160A and 160B, respectively. In one embodiment, the valves 150A and 150B comprise pinch valves. The injection system 10 controls the output valves 150A and 150B to control when fluid may be injected into a patient via the output tubing 100A and/or 100B. For example, the injection system 10 may open the valve 150A to allow the assembly 80A to inject fluid into the output tubing 100A. The system may also open the valve 150B to allow the assembly 80B to inject fluid into the output tubing 100B. The injection system 10 may close the valves 150A and 150B when the injection system 10 is filling fluid from the first reservoir 60 and/or second reservoir 40 into the corresponding syringe assembly. For example, the injection system 10 may close the valve 150A (and open valve 130A) when it fills fluid from the first reservoir 60 into the syringe assembly 80A.

Each valve 130A, 130B, 150A, 150B can utilize any suitable valve type including, for example, a ball valve, check valve, gate valve, piston valve, or similar fluid control feature. In one example, each valve can include a pinch valve, which can controllably pinch and release (e.g., compress and depress) a portion of compressible tubing (e.g., a portion compressible polymeric tubing) to control fluid communication through the tubing. In such an example, each pinch valve may be actuated by a solenoid, pneumatic actuator, or other form of drive mechanism.

FIG. 1C is a schematic diagram showing an example tubing configuration that may be used with injection system 10. As shown in FIG. 1C, an injector outlet tubing system 101 can be provided to deliver the fluids from the injector system 10 to the patient. In some embodiments, the tubing system includes the contrast media outlet tube 100A, the diluent output tubing 100B, and a patient line (e.g., catheter) 102. In certain embodiments, the outlet tubes and the patient line are connected via a selective fluid delivery valve 104. In such an embodiment, the tubing 100A may be coupled to a first input port of the selective fluid delivery valve 104, and the tubing 100B may be coupled to a second input port of the selective fluid delivery valve 104. An output port of the selective fluid delivery valve may further be coupled to the patient line 102. The selective fluid delivery valve can control the flow of fluid through the tubing 100A or 100B to the patient line 102, as will be described further below. As shown, a pressure sensor 105 can be associated with the selective fluid delivery valve. For example, the pressure sensor 105 can be in selective fluid and/or pressure communication with one or more of the lines fluidically connected to selective fluid delivery valve 104. The tubing system may also include other components as desired, such as additional inlet ports and tubes, purge ports and tubes, and additional valves, as desired. The injector outlet tubing system, or components thereof, may be intended for a single patient use (and as such may be replaceable), or may be intended for use with multiple patients (and as such may be permanently affixed to injection system 10).

Further, any or all of the various tubes in the tubing system can be continuous tubes or can include two or more tube segments joined together. In one embodiment, the output tubing 100A and 100B each includes a reusable portion and a single-use portion. In this embodiment, the single-use portions of the output tubing 100A and 100B may be coupled to the patient line 102 (optionally via selective fluid delivery valve 104) and discarded after a patient procedure. The reusable portions may be those portions of the tubing that are directly coupled to the outputs of the syringe assemblies 80A and 80B. The reusable portions and single-use portions may be coupled by fluid connectors, according to one embodiment.

FIGS. 2A and 2B are perspective views of certain embodiments of disposable fluid connections that may be used in a powered injection system, such as the injection system 10. FIG. 2A shows one embodiment in which a disposable fluid connector 190 may be used with the injection system 10. In this embodiment, the disposable fluid connector 190 may be loaded into an assembly portion 180 of the injection head 30. When assembled, the connector 190 is coupled as one single assembly to the injector. FIG. 2B shows an example of the connector 190 after it has been loaded into the assembly portion 180. The connector 190 may be used only for a single patient procedure (single use), according to one embodiment. In this embodiment, the connector 190 is discarded after is has been used for an injection procedure on a particular patient. In one embodiment, the connector 190 may be removably connected to the tubing 100A and 100B.

The connector 190 shown in FIGS. 2A and 2B includes output tubing 100A and 100B. The connector 190 further includes a connection 200. The connection 200 is configured to be coupled to the injection head 30. The connection 200 may be used to connect the connector 190 to an external medical device or to an external signal. For example, in one embodiment, the connection 200 may be connected to a pressure sensor, such that the injection system 10 may monitor hemodynamic signals. In this embodiment, an electrical cable may be used to connect the pressure sensor to the connection 200. The connection 200 can include a RJ-11 connector, and signals from the pressure sensor can then be routed to the injection system 10 via connection 200 for processing, as discussed further herein.

With further reference to FIGS. 1A and 1B, injection system 10 includes both a main control panel 20 and a small control panel 50. An operator of the injection system 10 may use the main control panel 20 to set up one or more parameters of an injection procedure prior to initiation of the procedure. The operator may also use the main control panel 20 to modify one or more aspects, parameters, etc. of an injection procedure during the procedure, or may also use the main control panel 20 to pause, resume, or end an injection procedure and begin a new procedure. The main control panel 20 can also display various injection-related information to the operator, such as flow rate, volume, pressure, rise time, procedure type, fluid information, and patient information. If included, the small control panel 50 can provide other functions or a subset of functions provided by the main control panel 20.

In use, the user (typically a physician or other caregiver) may enter safety parameters that will apply to the injection of radiographic contrast material into injection system 10. These safety parameters typically include the maximum amount of radiographic contrast material to be injected during any one injection, the maximum flow rate of the injection, the maximum pressure developed within a syringe body, and the maximum rise time or acceleration of the injection, or any other suitable information. After entering the appropriate control information, the user can activate injection system 10 to inject contrast material into a patient. Within the preset safety parameters, injection system 10 causes the flow rate of the injection to increase, in some cases, under the direct control of the user as the user depresses an injection trigger. In some examples, injection system 10 further injects a diluent into the patient after injecting contrast medium. When this occurs, the patient line (e.g., catheter) may be filled with a diluent instead of contrast media after an injection cycle.

The injection system 10 can be configured to perform a variety of operations. Representative operations include contrast fill, air purge, patient inject, saline flush, and patient pressure monitoring operations. In some examples, the injection system 10 also includes a pressure sensor testing protocol, as described further below.

Injection system 10 can be controlled using any suitable techniques, including an injector control system. In some examples, the injection system 10 includes a programmable processor (e.g., digital computer) which receives input signals from a user interface (e.g., a remote control, main control panel 20 (FIG. 1A), small control panel 50 (FIG. 1A)) and/or one or more pressure sensors (e.g., pressure sensor 105 in FIG. 1C). In these examples, the injection system 10 may provide control signals to control the display of, e.g., operation data, alerts, status information and user prompts, and/or control signals to control the motion of the plunger/pistons of syringe/plunger assemblies 80A and 80B through a motor drive circuit with a motor, and/or control signals to control the valves of injection system 10. The injector control system can be located in a variety of locations. In one embodiment, the injector control system can be physically located in the injection system 10. In another embodiment, the injection control system can be physically located outside of the injection system 10 and communicatively coupled to injection system 10 via a wired or wireless connection.

The injector control system can include a programmable processor and a storage device, which can store various software modules designed for specific functionality. The injector control system can also include a testing control module, as will be described further below. The software modules stored by the storage device can vary depending on a variety of factors, such as the kind of injector, the kind of injection process, and so on. Additional or different software modules other than those described herein can be stored by the storage device and/or the functionality of the various software modules can be modified to fit the particular application.

As briefly discussed above, it may be useful to monitor a patient's hemodynamic pressure during the operation of injection system 10. For these and other reasons, injection system 10 may include a pressure sensor (e.g., pressure sensor 105 in FIG. 1C) that is capable of monitoring the blood pressure of the patient. In such an example, the pressure sensor may be external to other injector hardware, or the pressure sensor may be integrated with other injector hardware. For instance, in one example, injection system 10 may include a pressure sensor in fluid and/or pressure communication with a patient line (e.g., patient line 102 in FIG. 1C) that can selectively monitor the patient's hemodynamic pressure pulse. When the patient line (e.g., a catheter) is in place in the patient, the pressure sensor can monitor the blood pressure of the patient through a column of fluid (e.g., contrast media, diluent) which extends from the patient line to the pressure sensor, thereby providing patient hemodynamic data to injection system 10. For example, a catheter inserted into the patient may hydraulically communicate pressure from within the body of the patient to a pressure sensor positioned outside of the body of the patient, which may then convert the hydraulic pressure to electrical signals for analysis, display, storage, processing, etc.

The pressure sensor can be any device suitable for sensing a hemodynamic pressure. In some embodiments, the pressure sensor is a pressure transducer that generates a signal as a function of the pressure imposed on the sensor. In such embodiments, the sensor may include a flexible membrane that moves in response to pressure changes in a fluid in contact with the membrane. The sensor then converts the sensed pressure to an electrical output signal (e.g., voltage and/or amperage) and transmits the signal to a pressure sensor control module. The signal from the pressure sensor may be further processed before it reaches the pressure sensor control module, or signal may be processed by the pressure sensor control module itself. Exemplary processing steps include amplification and analogue to digital conversation. In some embodiments, the pressure sensor is a pressure transducer rated to sense pressures between about −30 mmHg to about 300 mmHg. In such embodiments, the pressure sensor may be shielded from the higher pressures (e.g., about 6,200 mmHg) in the tubing experienced during a contrast injection operation.

Depending on the configuration of injection system 10, pressure sensor 105 may be in direct fluid communication with a patient line or indirect fluid communication with the patient line. For example, with reference to FIG. 1C, in one embodiment, pressure sensor 105 may be in fluid communication with diluent tubing 100B, which in turn is in selective fluid communication with patient line 102 through selective fluid delivery valve 104. When injection system 10 injects high pressure contrast media through output tubing 100A (e.g., by actuating selective fluid delivery valve 104 to close fluid communication between diluent tubing 100B and the patient line and to open fluid communication between contrast media tubing 100A and the patient line), the pressure sensor 105 can be isolated from the high pressure contrast media stream by selective fluid delivery valve 104, thereby protecting the pressure sensor from the forces of the high pressure stream. In another embodiment, pressure sensor 105 can be independently connected to selective fluid delivery valve 104 (e.g., through a separate tubing line not illustrated on FIG. 1C rather than through diluent tubing 100B). In this configuration, injection system 10 may separately place contrast media output tubing 100A, diluent tubing 100B, or tubing connected to pressure sensor 105 in fluid communication with patient line 102. Alternative configurations of pressure sensor 105 are both possible and contemplated. In certain embodiments, the pressure sensor has an associated stop-cock which allows it to be exposed to atmospheric pressure for calibration and also allows for removal/expulsion of any trapped air.

During some medical procedures, such as, for example, contrast media injections, liquid injection pressures can reach as high as 1200 lbs per square inch (psi) or more than 60,000 mm Hg. Pressure sensors (e.g., transducers) configured for physiological measurements generally cannot tolerate such high pressures while maintaining accuracy. For this reason, a pressure sensor in an injection system according to the disclosure may be isolated from a high-pressure fluid path during a high-pressure injection with a valve system. In many embodiments, the pressure sensor is positioned with respect to the selective fluid delivery valve (or incorporated therein) such that it is exposed to pressures on the low pressure diluent stream but is isolated from the higher pressures on the contrast media side. In exemplary embodiments, the selective fluid delivery valve can be used in connection with low pressure (e.g., less than 125 psi) to high pressure (e.g., greater than 1000 psi) medical fluid injections.

In an exemplary embodiment seen in FIG. 1C, a selective fluid delivery valve 104 has at least two, and in some examples three or more, ports that communicate with attached tubing. Such ports are, for example, a contrast media inlet port in fluid communication with contrast media output tubing 100A, a diluent inlet port in fluid communication with diluent outlet tube 100B, a pressure sensor port in fluid communication with pressure sensor 105, and a patient port (which may also be referred to as a valve outlet port) in fluid communication with a patient line (e.g., a catheter). Valves with other types of valve port configurations may be used. Exemplary configurations for a selective fluid delivery valve 104 can include a elastomeric-type valves as described in U.S. Pat. No. 7,617,837, the contents of which are incorporated by reference, and a manifold-type valve as described in US Patent Application Publication No. 2009/0149743.

FIG. 3 is a cross-sectional illustration of one example valve that may be used as selective fluid delivery valve 104. In the embodiment shown, the selective fluid delivery valve 104 comprises an elastomeric valve assembly that, in some examples, is part of a single-use kit. As seen in FIG. 3, selective fluid delivery valve 104 includes a valve body 220 that contains a diluent inlet port 230 (in fluid communication with a pressure sensor, not shown), a contrast media inlet port 240, and a patient/outlet port 250. In use, tubing 100B is connected to diluent inlet port 230, tubing 100A is connected to contrast media inlet port 240, and patient line 102 is connected to patient/outlet port 250. In some examples, as shown in the example of FIG. 3, contrast media inlet port 240 is tapered outward (in the forward flow direction, i.e., from right to left in FIG. 3) to create a pocket 245 in front of an elastomeric disc 260 so that as fluid travels through the contrast media inlet port 240 and into the empty pocket, air is forced from the pocket (purged) through the disc slit 270 and into the valve body 220 (more precisely, into the cavity in the valve body which is adapted to fluid flow). Thus, for example, in an angiographic procedure, as contrast media fills the empty pocket 245 of the disc holder 255 and pressure builds, the elastomeric valve disc 260 bends and eventually opens the slit 270 (which occurs at a certain known and pre-defined pressure) to inject fluid into the valve body.

Continuing with reference to FIG. 3, in an exemplary embodiment, valve body 220 has two internal tapers. A narrow taper 280 closest to the disc 260 that contains the diluent port, and a second wider taper 290. In operation, the narrow taper next to the disc 260 allows the diluent inlet port 230 to be sealed as pressure builds up and before fluid passes through the disc 260. The second, wider taper 290 and associated cavity create room for the disc to expand and allow the slit 270 to open fully.

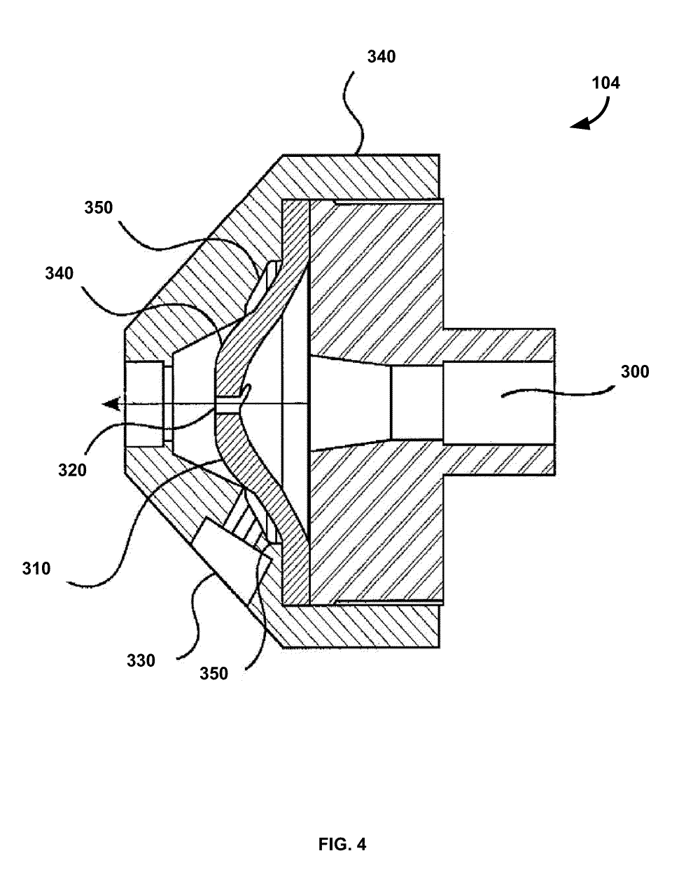

FIG. 4 depicts the exemplary selective fluid delivery valve of FIG. 3 in the high pressure fluid flow state (e.g., during a contrast inject operation). With reference to FIG. 4, contrast fluid under high pressure flows through inlet port 300 causing disc 310 to expand in the direction of flow (or to the left in FIG. 4), opening the disc slit 320. As the disc expands, a flap of the disc may cover the opening of the diluent port 330 in the cavity of the valve body 340. Further, the force maintained on the disc 340 by the incoming fluid may keep the diluent port shut during high pressure fluid flow, such as, for example, that experienced in a contrast fluid injection. In instances in which a pressure sensor is connected to a patient line via diluent port 330, the flap of the expanded elastomeric disc covering diluent port 330 may block fluid communication between the patient/outlet port (which may be filled with high pressure contrast media) and the pressure sensor, thereby shielding the pressure sensor. In this manner, the selective fluid delivery valve may be configured to fluidly seal a diluent inlet port, to which diluent output tubing 1 OOB and pressure sensor 105 are fluidly connected, during a high pressure contrast media injection.

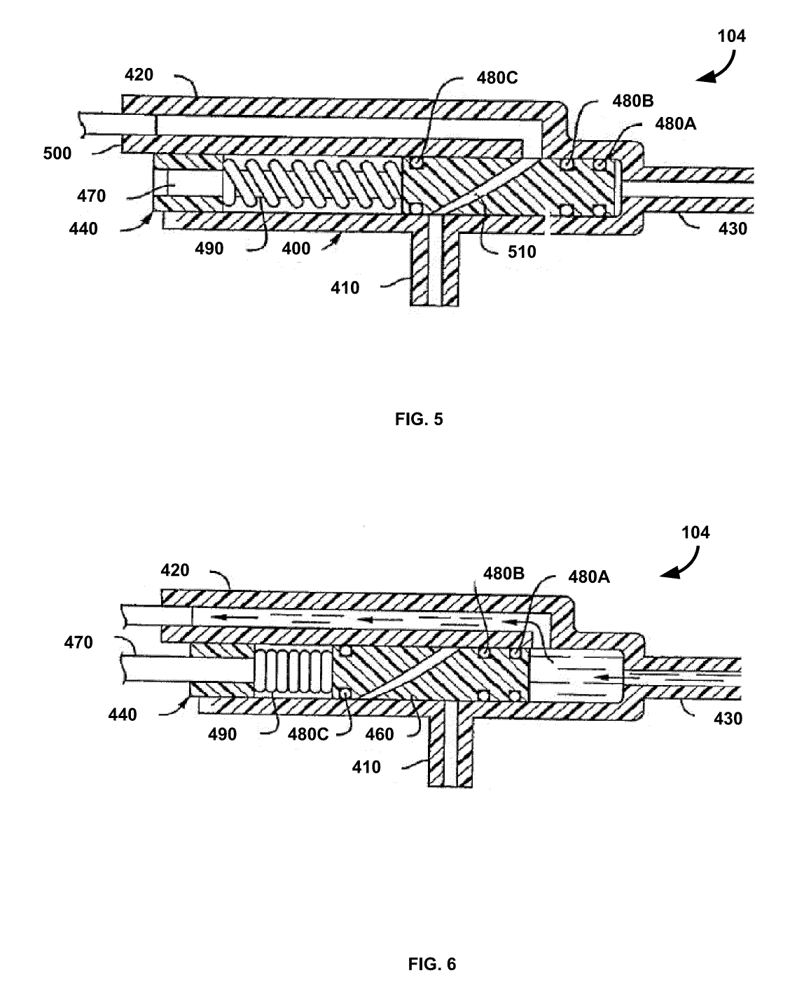

Another embodiment of a suitable selective fluid delivery valve 104 is shown in FIGS. 5 and 6. As shown in FIG. 5, selective fluid delivery valve 104 includes a spring biased spool valve which normally connects diluent port 410 (in fluid communication with a pressure sensor, not shown) and patient port 420. When radiographic contrast material is to be injected, the pressure of the radiographic material causes the spool valve to change states so that contrast media inlet port 430 is fluidly connected to patient port 420 instead of diluent port 410. In use, contrast media output tubing 100A is connected to diluent port 410, diluent tubing 100B is connected to contrast media inlet port 430, and patient line 102 is connected to patient port 420.

FIG. 5 illustrates selective fluid delivery valve 104 in its normal biased configuration (e.g., during a diluent inject operation). As shown, selective fluid delivery valve 104 contains spring loaded spool valve 440, which includes spool body 460, shaft 470, O-rings 480A, 480B and 480C, bias spring 490, and retainer 500. During a contrast fill operation, bias spring 490 urges spool body 460 to its right-most position (i.e., in the example configuration of FIG. 5). In this position, spool body 460 blocks contrast media inlet port 430 while connecting sensor diluent port 410 to patient port 420 through diagonal passage 510. O-rings 480A and 480B on the one hand, and O-ring 480C on the other hand, are positioned on the opposite sides of diagonal passage 510 to provide a fluid seal.

FIG. 6 illustrates the selective fluid delivery valve during a contrast media injection operation. The pressure at contrast media inlet port 430 has become sufficiently high to overcome the bias force of spring 490. Spool body 460 has been driven to the left so that contrast media inlet port 430 is connected to patient port 420. At the same time spool body 460 blocks diluent port 410. By virtue of the operation of spool valve 440, the high pressure generated by movement of shaft 470 is directly connected to patient port 420, while diluent port 410 and a pressure sensor in fluid communication with the diluent port are protected from the high pressure.

As briefly discussed above, a pressure sensor used to measure a hemodynamic pressure of a patient may have a maximum pressure above which the pressure sensor cannot be exposed without damaging the sensitivity and/or accuracy of the pressure sensor. For example, in the case of a medical pressure sensor used in a high pressure injection system, the pressure sensor may have a maximum pressure of less than 200 mm Hg (e.g., less than 175 mm Hg, less than 125 mm Hg). If exposed to pressures above the maximum pressure, the pressure sensor may be damaged, preventing the pressure sensor from monitoring the pressure of a patient and/or rendering pressure measurements generated by the pressure sensor unreliable. For this reason, a pressure sensor used in a high pressure injection system may be connected to a selective fluid delivery valve (e.g., FIGS. 4 and 5) to shield the pressure sensor from high pressure contrast media. However, if the selective fluid delivery valve is damaged during a high pressure contrast injection, not properly assembled during manufacturing, or exhibits other defects, the selective fluid delivery valve may not adequately shield the pressure sensor from high pressures.

To help ensure that a pressure sensor that may be exposed to pressures above a maximum exposure pressure has not been or will not be compromised in operation, the pressure sensor may be tested to ensure the operability and/or accuracy of the pressure sensor. In accordance with some examples described in this disclosure, a pressure sensor testing system may be used to test the operability and/or accuracy of a pressure sensor (e.g., pressure sensor 105 in injection system 10). In some examples, the test system can be included within a contrast injector system (e.g., injection system 10 in FIGS. 1A and 1B) or can be provided as a kit to be used apart from, or in conjunction with, a contrast injector system that does not include such a test system. Such a test system may be used to test the operability of a pressure sensor before, during, and/or after a medical procedure. In other examples, the test system may be used separately from a contrast injector system. For example, the test system may be used to test an assembly that includes a pressure sensor and a selective fluid delivery valve after manufacture of the assembly and/or after receipt of the assembly from a manufacture, but prior to incorporation of the assembly in a contrast injection system.

In some examples, the test system evaluates the operation of the pressure sensor before a high pressure contrast injection (or a simulated high pressure contrast injection) and after the high pressure contrast injection (or simulated high pressure contrast injection) and compares the performance of the pressure sensor before and after the high pressure contrast injection. For example, prior to passing a high pressure fluid (e.g., contrast injection media) through a selective fluid delivery valve connected to the pressure sensor (e.g., FIGS. 3-6), the test system may generate one or more pressure pulses having a predetermined magnitude and predetermined width (e.g., duration). The pressure pulses may communicate (e.g., hydraulically) to the pressure sensor, and the pressure sensor may convert the hydraulic pressure into electrical signals, which may be transmitted to the test system (e.g., for display and/or storage). The test system may cease generating pressure pulses and a high pressure contrast injection (or other high pressure fluid) may be passed through the selective fluid delivery valve connected to the pressure sensor. In some examples, as described above, the selective fluid delivery valve may close fluid communication between a port connected to the pressure sensor and an outlet port through which high pressure contrast injection media passes so as to shield the pressure sensor from the pressure of the contrast media.

After passing the contrast injection media through the selective fluid deliver valve connected to the pressure sensor, the pressure test system may again generate one or more pressure pulses having a predetermined magnitude and predetermined width. In some examples, the pressure pulses have the same magnitude and width as the pressure pulses generated before the high pressure contrast injection media was passed through the selective fluid delivery valve. The pressure pulses may communicate to the pressure sensor and, if the pressure sensor is still operable, the pressure sensor may generate electrical signals indicative of the received pressure pulses. If the pressure sensor does not generate and/or the test system does not receive any electrical signals in response to the generated pressure pulses, the test system may determine that the pressure sensor has failed, for example, due to a failure of the selective fluid delivery valve to shield the pressure sensor during the high pressure contrast injection. If the test system receives electrical signals from the pressure sensor in response to the generated pressure pulses, the test system may compare the electrical signals generated by the pressure sensor after the high pressure contrast injection to the electrical signals generated by the pressure sensor before the high pressure contrast injection. The pressure sensor may determine, based on the comparison, whether there has been any change in operation of the pressure sensor after the high pressure contrast injection as compared to before the high pressure contrast injection. For example, the test system may compare a magnitude of pressure determined by the pressure sensor before the high pressure contrast injection to the magnitude of pressure determined by the pressure sensor after the high pressure contrast injection. If the test system determines a difference between the pressures determined by the pressure sensor before and after the high pressure contrast injection (e.g., a different above or below a certain threshold), the difference may indicate that the pressure sensor was exposed to high pressure contrast injection media (e.g., due to a failure of the selective fluid delivery valve to shield the pressure sensor during the high pressure contrast injection). In this manner, the test system may help ensure the operability and/or accuracy of the pressure sensor for subsequent pressure monitoring.

FIG. 7 is a conceptual block diagram of one example test system 515 in accordance with the disclosure. In the example of FIG. 7, test system 515 includes pressure inducer 517, pressure generator 520, and testing control system 530. Pressure inducer 517 is configured (e.g., positioned and structured) to induce pressure changes in a tubing system (e.g., diluent output tubing 100B or patient/outlet line 102) in fluid communication with pressure sensor 105 via selective fluid delivery valve 104 during testing. Pressure generator 520 is connected to pressure inducer 517 via line 522, which, in different examples, can be an electrical line or a fluid line (e.g., a tube capable of delivering compressed air). Testing control system 530 controls test system 515. As shown, the testing control system 530 can be in electrical communication with the pressure sensor 105 via sensor line 532. Testing control system 530 can also be in electrical communication with the pressure generator 520 via generator line 534 and in electrical communication with the pressure inducer 517 via inducer line 536. Although test system 515 is illustrated in the example of FIG. 7 as being implemented in conjunction with injection system 10 (e.g., with contrast injection media tubing 100A and diluent output tubing 100B connected to first and second syringe/plunger assemblies 80A and 80B, respective), in other examples, the test system may be implemented separately from the injection system. For example, test system 515 may be a dedicated test system to which selective fluid delivery valve 104 and pressure sensor 105 can be connected for testing prior to being attached to injection system 10.

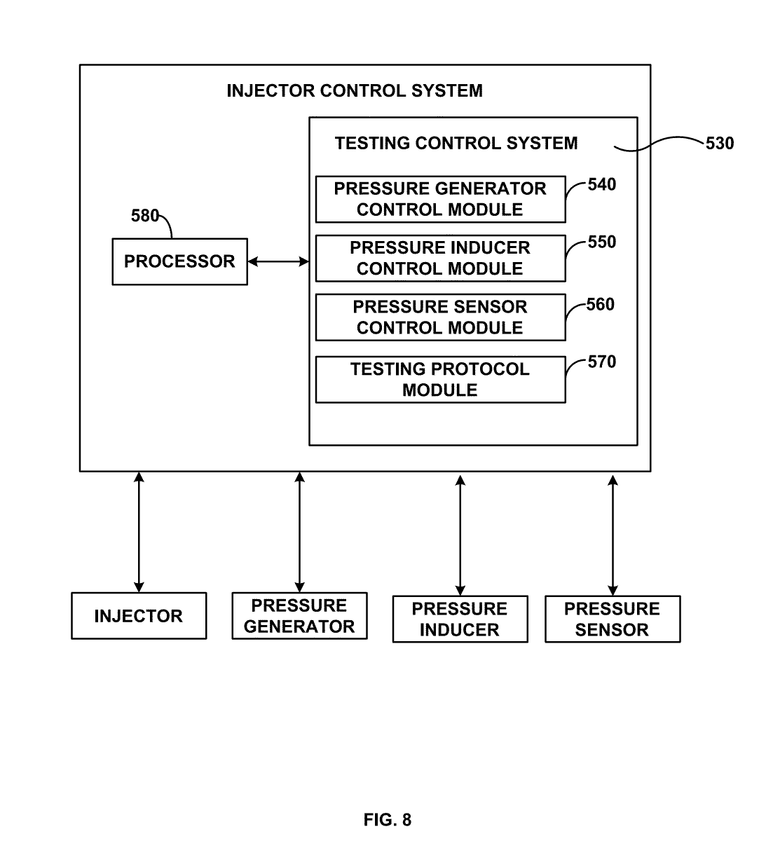

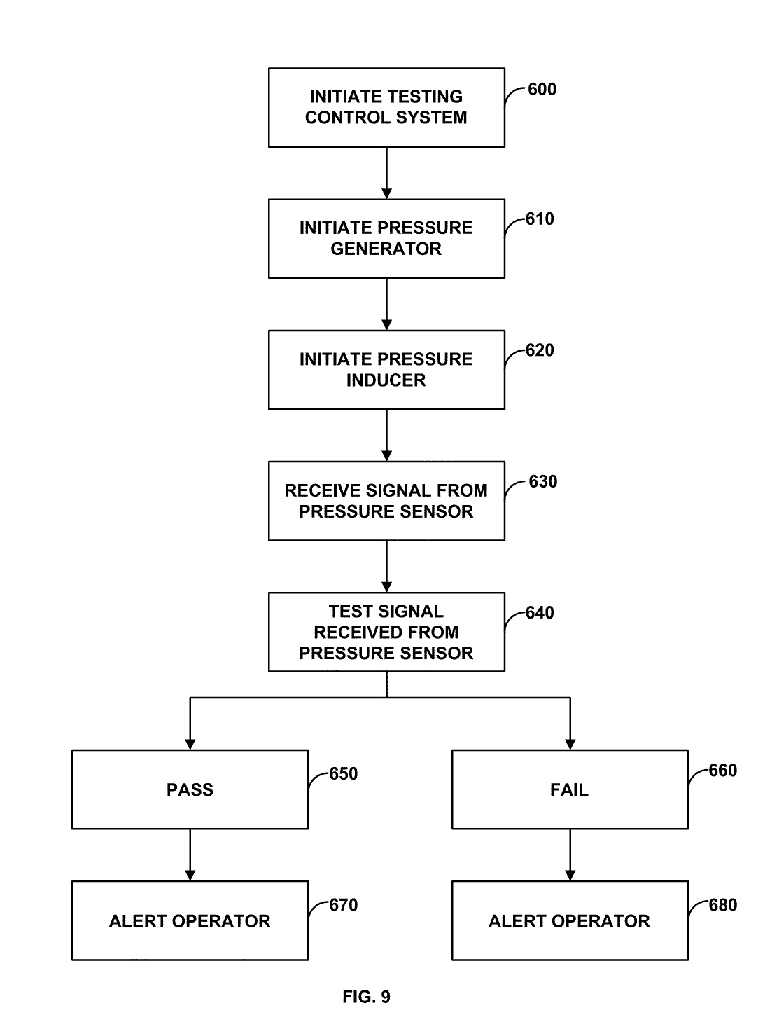

Testing control system 530 can include any suitable features for controlling test system 515. FIG. 8 is a conceptual block diagram illustrating some example features that may be included in a testing control system. As shown in FIG. 8, testing control system 530 can include one or more of pressure generator control modules 540, a pressure inducer control module 550, a pressure sensor data receiving module 560, and a testing protocol module 570. Testing control system 530 can include additional or different modules other than those illustrated in FIG. 8. Further, each of the different modules of testing control system 530 can be executed by a processor 580. In some embodiments, such as the embodiment shown in FIG. 8, the testing control system 530 is associated with an injector control system. In other embodiments, it is included a personal computer (e.g., with programmable Lab View software).

During operation, processor 580 can execute pressure generator control module 540 to generate control signals that are transmitted to the pressure generator. The control signals can control the pressure generator to generate a predetermined pressure that is supplied to the pressure inducer. Processor 580 can also execute pressure inducer control module 550 to generate control signals that are transmitted to the pressure inducer. Depending on the configuration of the pressure inducer, the control signals can control the pressure inducer to induce a series of pressures on the tubing. The pressure sensor data receiving module 560 can receive signals generated by the pressure sensor (e.g., in response to pressure induced by the pressure inducer). The testing protocol module 570 can analyze the signals received from the pressure sensor (e.g., via the pressure sensor data receiving module) and determine if the pressure sensor is operable according to a predetermined test or tests (e.g., data stored in memory).

Testing control system 530 can be implemented in a non-transitory computer-readable medium or storage device. The computer-readable medium can be an electronic, optical, magnetic, or other storage or transmission device capable of providing computer-readable instructions to a programmable processor. Examples of computer-readable media include a floppy disk, CD-ROM, magnetic disk, memory chip, ROM, RAM, an ASIC, a configured processor, all kinds of optical media, all kinds of magnetic tape or other magnetic media, or any other medium from which a computer processor can read instructions.

FIG. 9 shows an illustrative method that can be implemented in accordance with embodiments of the present invention. As shown in FIG. 9, some embodiments of the invention include the step of initiating the testing control module (600). This can be done through the main control panel 20 on an injector system or via a keyboard on a personal computer. After initiation, the testing control system (e.g., the pressure generator control module) can initiate the pressure generator (610). When there is sufficient pressure generated, the control system (e.g., the pressure inducer control module) can initiate the pressure inducer (620) to induce pressure on the tubing system. In some embodiments, the inducer will induce a series of pressure changes (e.g., a series of between about 20 and about 50 inductions) in the tubing system. In other embodiments, the inducer may only includes a single pressure change in the control system. The testing control system (e.g., the pressure sensor control module) can then receive a signal from the pressure sensor (630) and test the signal (640) (e.g., with the testing protocol module). The testing control system can then determine if the pressure sensor passes (650) or fails (660) according to any desired criteria.

Determinations can be made regarding subsequent actions based on whether the pressure sensor is operable or not. As shown in FIG. 9, the testing control system can include computer readable instructions for causing the testing control system to alert an operator (670) if the system determines that the pressure sensor passes (e.g., is operable). The testing control system can also alert an operator (680) if system determines that the pressure sensor fails (e.g., is inoperable). Optionally, other instructions may be incorporated into the computer-readable medium for causing the programmable processor to take subsequent actions, such as to disable an injector system if the pressure sensor fails the test until the operator gives an override command or replaces the inoperable pressure sensor.

In some embodiments, the testing system 515 cooperates with an injector control system 10. In such embodiments, the injector control system can instruct the injector system to complete one or more inject operations to fill the tubing system (of course, the tubing system can be filled with fluid by other methods). In some embodiments, a contrast inject operation may be performed to force contrast media into the patient line 102. After the contrast inject operation, the selective fluid delivery valve may revert back to allowing fluid communication between the diluent outlet tubing 100B and the patient line 102, thereby putting the contrast media in the patient line in fluid communication with the diluent outlet tubing and any pressure sensor in fluid communication with the diluent outlet tubing. Contrast injection media is generally more viscous than a diluent such as saline, which may dampen pressure pulses communicated through the tubing. Accordingly, in embodiments where the pressure inducer acts on a patient line filled with saline or another diluent, a better pressure signal may be obtained by the pressure sensor and transmitted to the test control system as compared to when the patient line is filled with contrast injection media. Accordingly, some embodiments include injecting contrast injection media into a tubing system and then flushing the tubing with saline or another diluent before initiating the pressure inducer to act on the tube.

The testing protocol module of the testing control system can be used to test the operability of the pressure sensor and/or the operability a selective fluid delivery valve associated with the pressure sensor. The operability of the features can be determined in any number of ways. In some embodiments, the pressure sensor is tested to determine if a patient's hemodynamic signals measured by the pressure sensor is substantially the same before and after injection operations. In other embodiments, the testing system determines if a selective fluid delivery valve associated with the pressure sensor has an appropriate recovery time after a high pressure injection operation. In yet other embodiments, the testing system can recognizes gross signal defects such as a baseline shift or low or lack of hemodynamic signals.

It can be determined if a patient's hemodynamic signals measured by the pressure sensor is substantially the same before and after injection operations in many ways. In some embodiments, the hemodynamic signals acquired from the pressure sensor before and after the high pressure injection operation are converted to a waveform and compared to make the determination.

In some embodiments, the amplitude of waveforms (e.g., peak to peak) generated both before and after a high pressure injection operation are compared. The testing system can indicate a “pass” if the difference is less than a predetermined variance threshold (e.g., within 10%) or a “fail” if the difference is more than a predetermined variance threshold (e.g., more than 10%).

In other embodiments, the testing system also tests for a baseline drift of the acquired waveforms. The testing system can compare the baseline of the hemodynamic signal acquired after a high pressure injection operation to a hemodynamic signal acquired before the high pressure inject operation. The testing system can indicate a “pass” if the difference is less than a predetermined variance threshold (e.g., within 5 mmHg) or a “fail” if the difference is more than a predetermined variance threshold (e.g., more than 5 mmHg).

In certain embodiments, the pressure sensor test system is useful for qualifying pressure sensors before the sensors are placed into service for a medical procedure. For example, a statistically significant portion of a batch of pressure sensors can selected for testing to determine if the remaining pressure sensors in the batch should be placed into service for a medical procedure. As another example, a pressure sensor intended to be used in a medical procedure can be tested at or near the location of its intended use. In either case, the pressure sensor can be tested by a testing protocol incorporated into an injector system, by a testing system kit, or by a testing system kit associated with an injector system that does not have a testing protocol incorporated therein.

In embodiments were the pressure sensor is associated with a selective fluid delivery valve, the valve and the sensor can be tested together. Such embodiments are useful for both testing the sensor itself and how the selective fluid delivery valve interacts with the pressure sensor (e.g., recovery time after a contrast inject protocol before the sensor accurately senses the patient's hemodynamic pressure).

In yet other embodiments, the pressure sensor can be tested one or more times during a medical procedure. Many procedures require several cycles of high pressure contrast injection. In some embodiments, the pressure sensor is tested after one, some, or all of the contrast injection phases. In some embodiments, the pressure sensor is tested by a testing protocol incorporated into an injector system. In other embodiments, the pressure sensor is tested by a testing system kit or by a testing system kit associated with an injector system that does not have an incorporated testing protocol.

The test system can include any suitable pressure inducer. In general, the pressure inducer will act on the tubing system to induce pressure changes to the fluid inside the tubing system. For example, where the tubing system is fabricated from a compressible material (e.g., a thermoplastic), the pressure inducer may act on the outside of the tubing to compress a portion of the tubing, thereby inducing a pressure change in the fluid inside the tubing. In some embodiments, the pressure inducer will induce pressures in the tubing system that mimic the pressures induced by a patient (i.e., pressures corresponding to a patient's blood pressure). In such embodiments, the pressure induced into a fluid within the tubing system will generally be between 50 and 200 mmHg such as, e.g., between 70 and 160 mmHg, although other pressures are possible.

A pressure inducer can be located at any desirable location, and the location may vary, e.g., depending on whether the pressure inducer is in a injector system, in a test kit, or in yet a different hardware package. In some embodiments, the pressure inducer is located downstream (i.e., on the patient side) of a selective fluid delivery valve. In other embodiments, the pressure inducer is located upstream (i.e., on the injector side) of the selective fluid delivery valve. In such embodiments, the pressure inducer can be located to induce pressures on the diluent outlet tube. In yet other embodiments, the pressure inducer is located within the contrast injector system (e.g., the pressure inducer can include valve 150B).

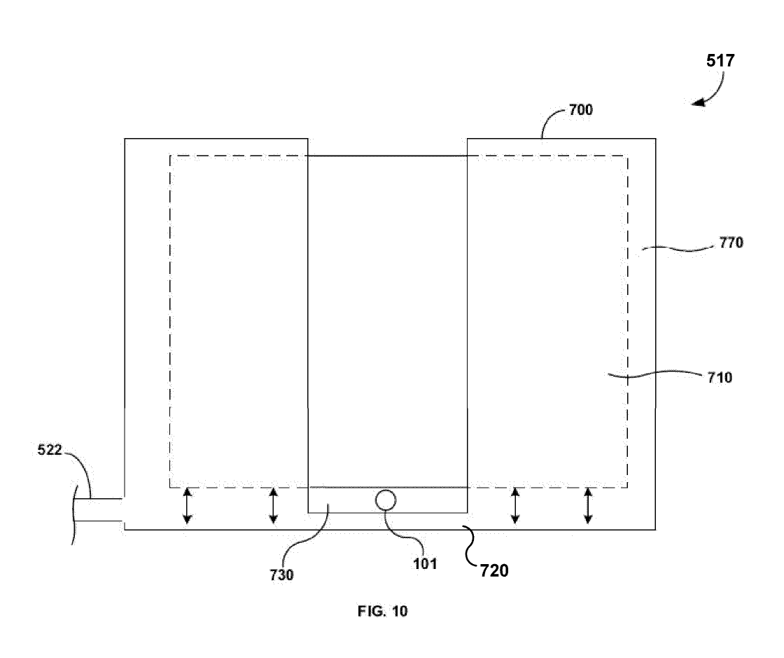

FIG. 10 is a conceptual cross-sectional view of one example pressure inducer 517. In the example of FIG. 10, pressure inducer 517 includes a pinch system 700 positioned to pinch the injector outlet tubing system 101 (e.g., diluent outlet tube or the patient line). The pinch system can include at least one pinching member 710 movable relative to a second (e.g., fixed) pinching member 720, with a section of injector outlet tubing system 101 positioned between the two pinching members. In the form exemplified in FIG. 10, the pinch system 700 includes a piston movable in response to pneumatic pressure changes supplied by air line 522. In this embodiment, the second pinching member 720 includes upwardly extending side that define a slot 730 sized to receive a portion of the injector outlet tubing system 101. In some examples, the second pinching member 720 also includes a corresponding slot (not shown) sized to receive a portion of the tubing system. In operation, the first pinching member 710 moves up and down relative to second pinching member 720 in response to pressurized air supplied via air line 522. The injector outlet tubing system 101 during a downward stroke of first pinching member 710 and release the pinch during an upward stroke of first pinching member 710, thereby inducing pressure pulses within injector outlet tubing system 101.

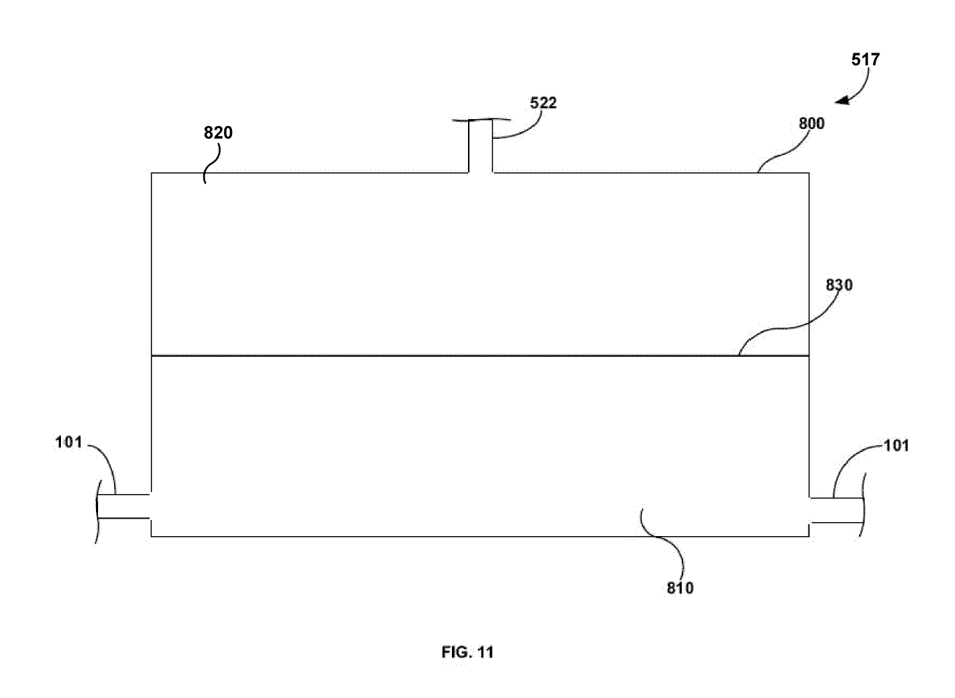

FIG. 11 is a conceptual cross-sectional view of another example pressure inducer 517 that includes a flexible membrane. In the example of FIG. 11, pressure inducer 517 includes a divided flexible system 800 comprising a wet side 810, a dry side 820, and a divider 830 separating the wet side from the dry side (e.g., hermetically sealing the wet side from the dry side). When configured as shown in FIG. 11, the injector outlet tubing system 101 can be in fluid communication with the wet side, and a pneumatic pressure generator can be in pneumatic communication with the dry side via line 522. During operation, pressure supplied via line 522 can act against the divider, which acts as a diaphragm, to impart the pressure on the wet side, which in turn communicates the pressure to the tubing system and ultimately the pressure sensor.

Independent of the specific configuration of pressure inducer 517, the pressure inducer can be powered or actuated by any suitable pressure generator. For example, the pressure inducer can be powered or actuated by a pressure generator including a pneumatic system (e.g., a compressor capable of generating compressed air at a pressure of between about 50 and 150 psi (e.g., 100 psi)), an electromechanical system, or yet other systems. In some examples, such as some examples that include a pinch system, the pinch system can be powered by compressed air generated by a compressor. In such embodiments, compressed air is supplied to the pinch system to move the at least one moveable pinching member with respect to the second pinching member to induce pressures in the tubing system. In other examples, such as other examples that include a divided flexible system, the flexible divider can be biased with compressed air that is supplied by a compressor. The compressor can supply the compressed air to the dry side of the divided flexible system, thereby causing the divider to act against the fluid on the wet side of the divider that is in fluid communication with the tubing system.

In other embodiments, as noted above, a pressure generator can include an electromechanical device or other device. For example, a pressure generator can include a solenoid. In embodiments including a pinch system, the solenoid can move at least one movable pinching member with respect the second pinching member to induce pressures in the tubing system.

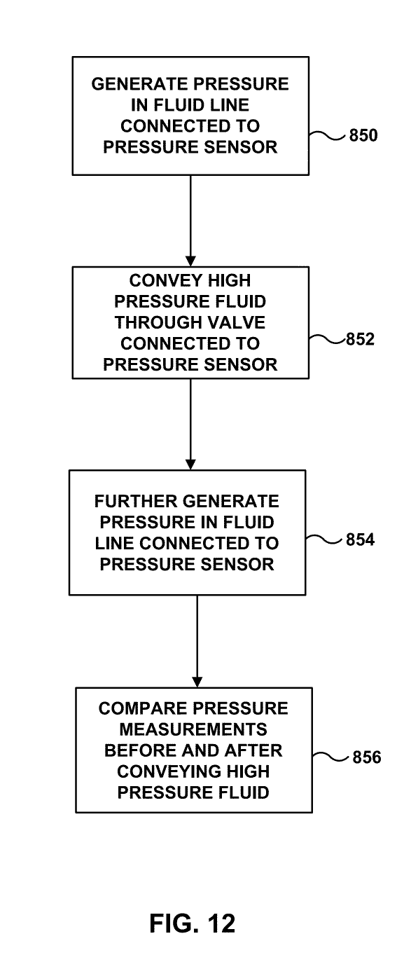

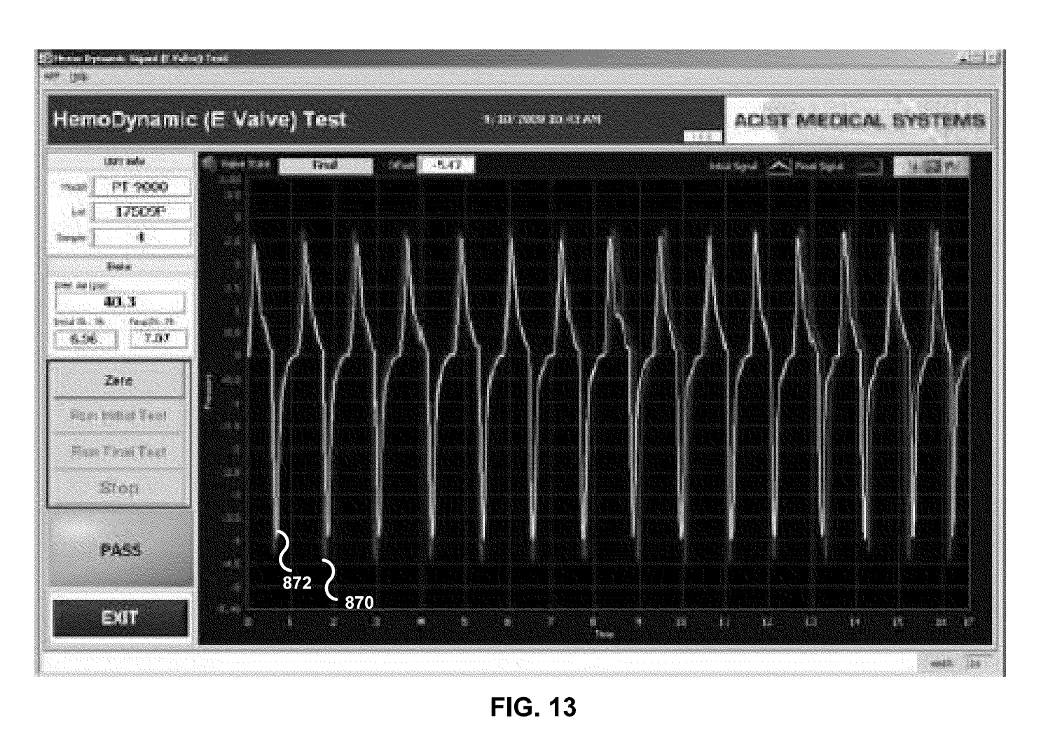

FIG. 12 is a flow chart showing another example method for testing a pressure sensor. In particular, FIG. 12 illustrates an example method for testing a medical pressure sensor coupled to a selective fluid delivery valve, where the selective fluid delivery valve is configured to shield the medical pressure sensor from pressurized fluid at a pressure above a maximum operating pressure of the pressure sensor. For ease of description, the method of FIG. 12 is described with respect to selective fluid delivery valve 104 (FIGS. 3-6) and pressure sensor 105. In other examples, the method of FIG. 12 may be performed on using valves and pressure sensors having other configurations.

The technique of FIG. 12 includes generating one or more pressure pulses in a fluid line fluidly coupled to pressure sensor 105 (850). In some examples, pressure sensor 105 is coupled to a compressible fluid line (e.g., a thermoplastic fluid line), and generating pressure pulses includes compressing a portion of the fluid line. For example, under the control of a processor (e.g., processor 580), a pressure inducer may press on a portion of a compressible fluid line to cause a pressure change in the fluid line. When the fluid line is filled with a liquid fluid (e.g., contrast injection media, diluent), compression of the fluid line may pressurize the liquid fluid and cause a pressure wave to transfer through the liquid to pressure sensor 105. Pressure sensor 105 may receive the pressure wave and generate an electrical signal indicative of the magnitude and/or duration of the pressure applied to the compressible fluid line. The electrical signal generated by the pressure sensor may provide a first pressure reading (or set of pressure readings) that may be transmitted to a processor and stored on a computer readable medium, displayed, and/or otherwise processed.

In different examples, generating one or more pressure pulses in a fluid line fluidly coupled to pressure sensor 105 (850) involves generating a single pressure pulse or generating a plurality of pressure pulse in the fluid line. In general, increasing the number of pressure pulses may increase the number of pressure readings provided by pressure sensor 105, which, in turn, may provide more data for subsequent analysis. A single pressure pulse may be generated by compressing a portion of a compressible fluid line once and then releasing the compression so that the compressible fluid line returns to its original size and/or shape. Multiple pressure pulses may be generated by repeating the cycle of compression and release multiple time. In some examples, generating one or more pressure pulses in a fluid line fluidly coupled to pressure sensor 105 (850) involves generating at least 10 pressure pulses in the fluid line such as, e.g., at least 20 pressure pulses, at least 25 pressure pulses, or at least 50 pressure pulses.

Although the magnitude of the pressure pulses generated in the fluid line fluidly coupled to pressure sensor 105 (850) may vary, the pressure pulses will typically be below the maximum operating pressure of the sensor. The magnitude of the pressure pulses generated in the fluid line may be adjusted by adjusting the amount of force with which a pressure inducer presses on a portion of a compressible fluid line. For example, pressing on the compressible fluid line with a first amount of force may generate a pressure pulse having a first magnitude, while pressing on the compressible fluid line with a second amount of force greater than the first amount of force may generate a pressure pulse having a second magnitude greater than the first amount of force. In some examples, the pressure pulses generated in the fluid line are less than approximately 200 mm Hg such as, e.g., from approximately 25 mm Hg to approximately 200 mm Hg, or from approximately 70 mm Hg and approximately 160 mm Hg. When multiple pressure pulses are generated in the fluid line, each pressure pulse may be of the same magnitude, or at least one pressure pulse may have a magnitude different than at least one other pressure pulse.

The technique of FIG. 12 also includes conveying high pressure fluid through the selective fluid delivery valve 104 to which pressure sensor 105 is fluidly connected (852). Under the control of a processor (e.g., processor 580), a pressure inducer may cease applying pressure to a compressible portion of fluid line so as to stop generating pressure pulses in the fluid line. The processor may then control a high pressure fluid source (e.g., injection system 10) to convey high pressure fluid through selective fluid delivery valve 104. The fluid may be contrast injection media, diluent, or another fluid. In general, the fluid may be at a pressure greater than the maximum operating pressure of pressure sensor 105. For instance, in some examples, the fluid is at a pressure greater than 100 psi such as, e.g., greater than 500 psi, greater than 1000 psi, or greater than 1200 psi. The selective fluid delivery valve 104 may shield pressure sensor 105 from the high pressure fluid as the high pressure fluid is conveyed through the selective fluid delivery valve (852). For example, introduction of high pressure fluid into selective fluid delivery valve 104 may move an internal portion of the valve so as to block fluid communication between a valve port fluidly connected to pressure sensor 105 and the high pressure fluid. In some examples, conveying high pressure fluid through the selective fluid delivery valve 104 (852) includes conveying high pressure contrast injection media through the selective fluid delivery valve followed by conveying comparatively lower pressure diluent (e.g., saline) through the selective fluid delivery valve. In such examples, the fluid line fluidly coupled to pressure sensor 105 may be filled with diluent after conveying high pressure fluid through the selective fluid delivery valve 104 (852).

Subsequent to conveying high pressure fluid through the selective fluid delivery valve 104 to which pressure sensor 105 is fluidly connected (852), the technique of FIG. 12 includes further generating one or more pressure pulses in the fluid line fluidly coupled to pressure sensor 105 (854). Under the control of a processor, the pressure inducer may again press on a portion of the compressible fluid line connected to pressure sensor 105. The pressure sensor may generate a single pressure pulses or multiple pressure pulses, where each pressure pulse has any suitable magnitude, as discussed above with respect to step (850). In some examples, each pressure pulse generated in the fluid line fluidly coupled to pressure sensor 105 (854) is of the same magnitude as each pressure pulse generated in the fluid line before conveying the high pressure fluid through the selective fluid delivery valve (850). Further, while the number of pressure pulses generated in the fluid line fluidly coupled to pressure sensor 105 after conveying a high pressure fluid through the selective fluid delivery valve 104 (854) can vary, in some examples, at least 10 pressure pulses are generated in the fluid line such as, e.g., at least 20 pressure pulses, at least 25 pressure pulses, or at least 50 pressure pulses. Each pressure pulse generated in the fluid line fluidly connected to pressure sensor 105 may cause pressure wave to transfer through the liquid to pressure sensor 105. Pressure sensor 105 may receive the pressure wave and generate an electrical signal indicative of the magnitude and/or duration of the pressure applied to the compressible fluid line for each pressure pulse. The electrical signal generated by the pressure sensor may provide a second pressure reading (or set of pressure readings) that may be transmitted to the processor and stored on a computer readable medium, displayed, and/or otherwise process.

The technique of FIG. 12 includes comparing the measurements made by pressure sensor 105 before conveying high pressure fluid through selective fluid delivery valve 104 (e.g., the first pressure reading or set of pressure readings) to measurements made by the pressure sensor after conveying high pressure fluid through the selective fluid delivery valve (e.g., the second pressure reading or set of pressure readings) (856). For example, a processor (e.g., processor 580) may compare pressure measurements made by pressure sensor 105 before conveying high pressure fluid through selective fluid delivery valve 104 (e.g., the first pressure reading or first set of pressure readings) to pressure measurements made by the pressure sensor after conveying high pressure fluid through the selective fluid delivery valve (e.g., the second pressure reading or second set of pressure readings) and determine if there is any difference in the pressure measurements. In some instances in which the processor compares a first set of pressure readings to a second set of pressure readings, the processor may omit the initial pressure reading(s) associated with the first set of pressure readings and/or the initial pressure reading(s) second set of pressure readings from the comparison. For example, the processor may omit the initial pressure reading(s) associated with approximately the first to approximately the tenth pressure pulses used to generate the first set of pressure readings and initial pressure reading(s) associated with approximately the first to approximately the tenth pressure pulses used to generate the second set of pressure readings from the data comparison. The fluid tubing fluidly connected to pressure sensor 105 may expand or otherwise deform during these initial pressure pulses, rendering the pressure readings associated with the pressure pulses less reliable than subsequent pressure pulses. In other examples, the processor does not omit initial pressure readings from the comparison.

In one example, the processor determines a maximum and/or a minimum pressure measured by pressure sensor 105 while generating pressure pulses in the fluid line fluidly coupled to pressure sensor 105 (850) and also determines a maximum and/or minimum pressure measured by the pressure sensor while generating pressure pulses in the fluid line after high pressure fluid injection (854). The processor can then compare the maximum and/or minimum pressure measured by the pressure sensor before high pressure fluid injection to the maximum and/or minimum pressure measured by the pressure sensor after high pressure fluid injection, for example, by determining a difference between the maximum and/or minimum measured before high pressure injection to the maximum and/or minimum measured after high pressure injection.

In another example, the processor determines a difference between the maximum and minimum pressure measured by pressure sensor 105 while generating pressure pulses in the fluid line fluidly coupled to pressure sensor 105 (850) and also determines a difference between the maximum and minimum pressure measured by the pressure sensor while generating pressure pulses in the fluid line after high pressure fluid injection (854). The difference between the maximum and minimum pressures determined by the processor may be a peak-to-peak difference. The processor can then compare the peak-to-peak pressure difference measured by the pressure sensor before high pressure fluid injection to the peak-to-peak pressure difference pressure measured by the pressure sensor after high pressure fluid injection, for example, by subtracting one peak-to-peak pressure difference from the other peak-to-peak pressure difference.

In instances in which multiple pressure pulses are generated in the fluid line fluidly connected to pressure sensor 105 in accordance with this example, the processor may determine a difference between a maximum pressure measured by the pressure sensor across all pressure pulses (e.g., either before or after high pressure fluid injection) and a minimum pressure measured by the pressure sensor across all pressure pulses (e.g., either before or after high pressure fluid injection) to determine the peak-to-peak difference. Alternatively, the processor may determine a difference between a high pressure measured by the pressure sensor and a low pressure measured by the pressure sensor for each pressure pulse. The processor may then determine a composite peak-to-peak difference which, in different examples, may be an average (e.g., mean, median) peak-to-peak difference based on multiple peak-to-peak difference associated with each of the multiple pressure pulse, a greatest or a least peak-to-peak difference from the multiple peak-to-peak difference associated with each of the multiple pressure pulses, or any other suitable composite peak-to-peak difference.

In still another example, the processor determines a difference between an average (e.g., mean, median) pressure measured by pressure sensor 105 while generating pressure pulses in the fluid line fluidly coupled to pressure sensor 105 before high pressure fluid injection (850) and also determines an average pressure measured by the pressure sensor while generating pressure pulses after high pressure fluid injection (854). The average pressure determined by the processor may be a baseline pressure. The processor can then compare the baseline pressure measured by the pressure sensor before high pressure fluid injection to the baseline pressure measured by the pressure sensor after high pressure fluid injection, for example, by subtracting one baseline pressure from the other baseline pressure difference.

In some examples, the processor determines pressure sensor 105 (and valve 104) has “passed” or “failed” by comparing the determined differences between pressure measurements made before high pressure fluid injection and after high pressure fluid injection to one or more thresholds stored in a computer readable memory. For example, the processor may compare a difference between maximum pressures, minimum pressures, peak-to-peak pressures, and/or baseline pressures before and after high pressure fluid injection to one or more thresholds stored in a computer readable memory. If the processor determines that the pressure difference is within the one or more thresholds, the processor may determine that pressure sensor 105 (and/or valve 104) has “passed,” while if the processor determines that the pressure difference is outside of the one or more thresholds, the processor may determine that pressure sensor 105 (and valve/or 104) has “failed.

In some examples, if the pressure difference before and after high pressure contrast injection is less than a certain percentage, the processor may determine that pressure sensor 105 (and valve 104) has “passed” testing. For example, if the pressure measured after high pressure fluid injection is less than ±25% of the pressure measure before high pressure fluid injection such as, e.g., less than ±15%, less than ±10%, or less than ±5% of the pressure measure before high pressure fluid injection, the processor may determine that pressure sensor 105 (and valve 104) has “passed” testing. If the processor determines that the pressure measured after high pressure fluid injection is greater than or equal to any of the foregoing values before high pressure fluid injection, the processor may determine that pressure sensor 105 (and valve 104) has “failed” testing. In other examples, if the pressure difference before and after high pressure contrast injection is less than a certain value, the processor may determine that pressure sensor 105 (and valve 104) has “passed” testing. For example, if the pressure measured after high pressure fluid injection is less than ±20 mm Hg of the pressure measure before high pressure fluid injection such as, e.g., less than ±15 mm Hg, less than ±10 mm Hg, less than ±5 mm Hg, or less than ±3 mm Hg of the pressure measure before high pressure fluid injection, the processor may determine that pressure sensor 105 (and valve 104) has “passed” testing. If the processor determines that the pressure measured after high pressure fluid injection is greater than or equal to any of the foregoing values before high pressure fluid injection, the processor may determine that pressure sensor 105 (and valve 104) has “failed” testing.

FIGS. 13-15 are plots illustrating example pressures that may be measured by pressure sensor 104 before a high pressure contrast injection and after a high pressure contrast injection according to the example method of FIG. 12. In each of the example plots of FIGS. 13-15, the X-axis is time and the Y-axis is the magnitude of pressure measured by pressure sensor 105. FIG. 13 illustrates a pressure 870 measured by pressure sensor 105 before a high pressure contrast injection and a pressure 872 measured by pressure sensor 105 after a high pressure contrast injection. Pressure 870 and pressure 872 are centered around substantially the same baseline pressure and exhibit a peak-to-peak difference that, in some examples, is within a tolerance threshold of pressure sensor 105. In these examples, pressure sensor 105 (and/or valve 104) may be designated as having “passed” testing.

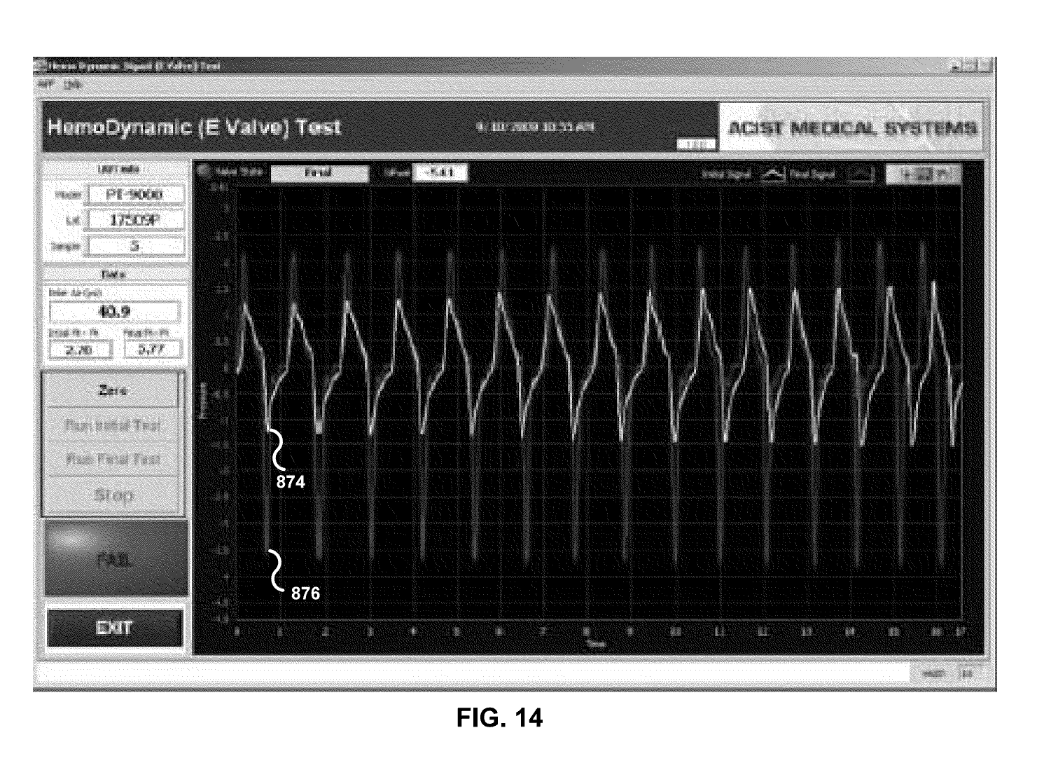

FIG. 14 illustrates a pressure 874 measured by pressure sensor 105 before a high pressure contrast injection and a pressure 876 measured by pressure sensor 105 after a high pressure contrast injection. Pressure 874 is centered around substantially the same baseline as pressure 876. However, pressure 874 exhibits lower maximum pressure peaks, lower minimum pressure peaks, and a smaller peak-to-peak distance than pressure 876. In some examples, the difference in maximum pressure, minimum pressure, and/or peak-to-peak pressure between pressure 874 and pressure 876 may be outside of a tolerance threshold set for pressure sensor 105. In these examples, pressure sensor 105 (and/or valve 104) may be designated as having “failed” testing.

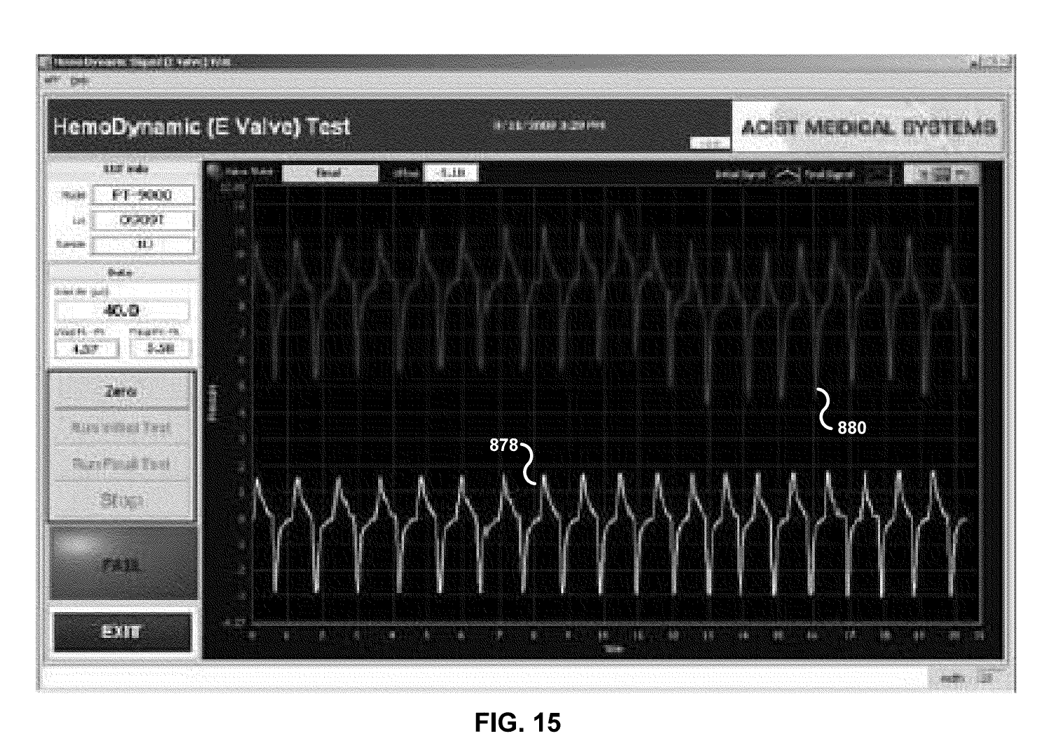

FIG. 15 illustrates a pressure 878 measured by pressure sensor 105 before a high pressure contrast injection and a pressure 880 measured by pressure sensor 105 after a high pressure contrast injection. Pressure 878 exhibits substantially the same peak-to-peak pressure difference as pressure 880 (e.g., within a tolerance threshold). However, pressure 878 is centered about a lower baseline pressure than pressure 880. In some examples, the difference between the baseline pressure of pressure 878 and the baseline pressure of pressure 880 may be outside of a tolerance threshold set for pressure sensor 105. In these examples, pressure sensor 105 (and/or valve 104) may be designated as having “failed” testing.

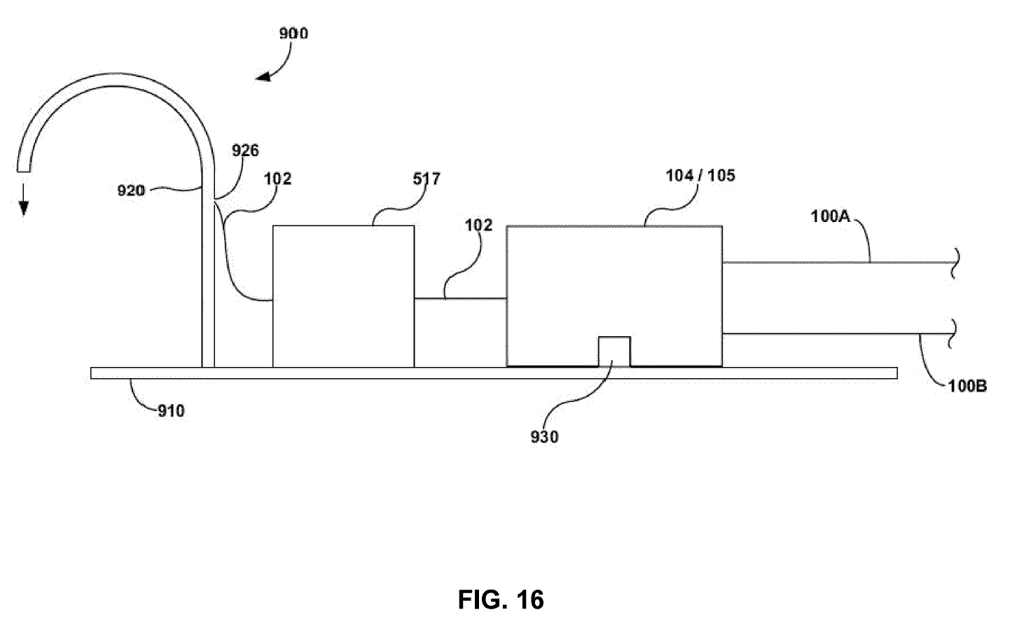

A test system in accordance with the disclosure can assume a variety of different physical configurations. In some configurations, the test system includes a stand to hold and elevate a portion of a tubing system during testing. FIG. 16 is a schematic diagram of one such example a test system in accordance with an embodiment of the invention. In the example of FIG. 16, a stand 900 is provided to hold a portion of the tubing system. Such a stand may be useful for comparing a single pressure sensor over time because the stand may eliminate or reduce pressure fluctuations caused by a relative height changes between the patient line and the pressure sensor. In some embodiments, the stand includes a base 910 and an arm 920 extending above the base. The arm includes a tube holder to hold a portion of the tubing system at a fixed height. In some embodiments, the arm has an aperture 926 that allows the patient line to be inserted in to the arm. As shown, the stand may also include a selective fluid delivery valve clip 930 to retain the selective fluid delivery valve. Further, as shown, the pressure inducer 517 may also be placed on (and optionally fixed to) the stand. In this manner, stand 900 may provide a fixed height differential between patient line 102 and pressure sensor 105.

It should be noted that for a test during a medical procedure, when the catheter is inserted into a patient, such a stand may not be needed. In some examples, pressure sensor 105 is maintained at a substantially constant height (e.g., relative to a patient) when generating pressure readings before a high pressure contrast injection and when generating pressure readings after the high pressure contrast injection.

In the foregoing detailed description, the invention has been described with reference to specific embodiments. However, it may be appreciated that various modifications and changes can be made without departing from the scope of the invention as set forth in the appended claims. Thus, some of the features of preferred embodiments described herein are not necessarily included in preferred embodiments of the invention which are intended for alternative uses.

1. A powered injection system to deliver contrast injection media to a patient, comprising:
a control panel to receive input from a user;
an injector control system having a pressure sensor testing control system in electrical communication with the control panel;
a syringe driven by a power source in electrical communication with the control panel;
a first fluid reservoir in fluid communication with the syringe;
a syringe outlet tube in fluid communication with the syringe;
a second fluid reservoir;
a tubing system to deliver a first fluid from the first fluid reservoir to a patient line and to deliver a second fluid from the second fluid reservoir to the patient line;
a selective fluid delivery valve in fluid communication with the syringe outlet tube and the second fluid reservoir and having a valve outlet port in fluid flow communication with the patient line, the selective fluid delivery valve being selectively positionable to provide a fluid flow path between the syringe outlet tube and the valve outlet port or between the second fluid reservoir and the valve outlet port;
a pressure sensor configured to monitor a homodynamic pressure of the patient, the pressure sensor being in fluid flow communication with the patient when the selective fluid delivery valve is positioned to provide fluid flow communication between the second fluid reservoir and the patient line and not being in fluid flow communication with the patient when the selective fluid delivery valve is positioned to provide fluid flow communication between the first fluid reservoir and the patent line, the pressure sensor being in electrical communication with the injector control system; and
a pressure inducer in electrical communication with the injector control system, the pressure inducer positioned to induce a pressure on the tubing system when the pressure sensor is in fluid flow communication with the patient line to generate a test pressure signal to test an operability of the pressure sensor.

2. The powered injection system of claim 1, wherein the selective fluid delivery valve is a manifold valve.

3. The powered injection system of claim 1, wherein the selective fluid delivery valve is an elastomeric valve.

4. The powered injection system of claim 1, wherein the pressure inducer is a pinch valve.

5. The powered injection system of claim 1, wherein the first fluid is a contrast injection media.

6. The powered injection system of claim 1, wherein the second fluid is saline.

7. The powered injection system of claim 1, wherein the pressure sensor is a transducer.

8. The powered injection system of claim 1, further including a pressure generator.

9. The powered injection system of claim 8, wherein the pressure generator includes an air compressor.

10. A test system for testing a hemodynamic pressure sensor, the test system comprising:
a pressure inducer configured to induce pressure changes to a tubing system in fluid communication with the hemodynamic pressure sensor;
a pressure generator configured to power the pressure inducer; and
a testing control system in electrical communication with the hemodynamic pressure sensor, the pressure inducer, and the pressure generator.

11. The test system of claim 11, wherein the testing control system is associated with an injector control system.

12. The test system of claim 11, wherein the testing control system is associated with a personal computer.

13. A method of testing a hemodynamic pressure sensor, the method comprising:
initiating a testing control system;
initiating a pressure generator;
initiating a pressure inducer to induce pressure to a tubing system associated with the hemodynamic pressure sensor;
receiving a signal from the hemodynamic pressure sensor in response to the induced pressure;
analyzing the signal received from the hemodynamic pressure sensor; and
determining if the hemodynamic pressure sensor passes or fails.

14. The method of claim 13, further including filling the tubing system with a fluid before inducing pressure.

15. The method of claim 14, wherein the tubing system includes a contrast media outlet tube filled with contrast media fluid, a diluent outlet tube filled with diluent, and a patient line filled with diluent, and initiating the pressure inducer to induce pressure to the tubing system comprises initiating the pressure inducer to compress the patient line filled with diluent.

16. A method comprising:
generating at least one pressure pulse in a fluid line fluidly connected to a medical pressure sensor so as to generate a first pressure reading, wherein the medical pressure sensor is fluidly connected to a valve that is configured to shield the medical pressure sensor from a high pressure fluid injection;
conveying high pressure fluid through the valve fluidly connected to the medical pressure sensor, the high pressure fluid being at a pressure above a maximum operating pressure of the medical pressure sensor;
subsequent to conveying the high pressure fluid, generating at least one pressure pulse in the fluid line fluidly connected to the medical pressure sensor so as to generate a second pressure reading;
comparing the first pressure reading to the second pressure reading; and
determining based on the comparison whether the medical pressure sensor has passed or failed.

17. The method of claim 16, wherein generating at least one pressure pulse in the fluid line fluidly connected to the medical pressure sensor so as to generate the first pressure reading comprises generating a first plurality of pressure pulses in the fluid line fluidly connected to the medical pressure sensor so as to generate a first set of pressure readings, and generating at least one pressure pulse in the fluid line fluidly connected to the medical pressure sensor so as to generate the second pressure reading comprises generating a second plurality of pressure pulses in the fluid line fluidly connected to the medical pressure sensor so as to generate a second set of pressure readings.

18. The method of claim 17, wherein a magnitude of each pressure pulse in the first plurality of pressure pulses is the same as a magnitude of each pressure pulse in the second plurality of pressure pulses.

19. The method of claim 18, wherein the magnitude of each pressure pulse in the first plurality of pressure pulses and the magnitude of each pressure pulse in the second plurality of pressure pulses ranges from approximately 25 mm Hg to approximately 200 mm Hg.

20. The method of claim 16, wherein the pressure of the high pressure fluid is greater than 5000 mm Hg.

21. The method of claim 16, wherein comparing the first pressure reading to the second pressure reading comprises determining a first peak-to-peak pressure difference for the first pressure reading, determining a second peak-to-peak pressure difference for the second pressure reading, and determining a difference between the first peak-to-peak pressure difference and the second peak-to-peak pressure difference.

22. The method of claim 16, wherein comparing the first pressure reading to the second pressure reading comprises determining a first baseline pressure for the first pressure reading, determining a second baseline pressure for the second pressure reading, and determining a difference between the first baseline pressure and the second baseline pressure.
